(19) United States
(12) Patent Application Publication
(10) Pub. No.: US 2012/0101255 A1
(43) Pub. Date: Apr. 26, 2012

(54) PEPTIDE-BASED REGULATION OF GAP JUNCTIONS

(75) Inventors: Mario Delmar, Syracuse, NY (US); Steven M. Taffet, Syracuse, NY (US); Bjarne Due Larsen, Roskilde (DK)

(73) Assignees: Zealand Pharma A/S, Glostrup (DK); The Research Foundation of State University of New York, Albany, NY (US)

(21) Appl. No.: 13/279,703

(22) Filed: Oct. 24, 2011

Related U.S. Application Data
(62) Division of application No. 12/172,355, filed on Jul. 14, 2008, now abandoned.
Application No. 12/172,355 is a continuation-in-part of application No. PCT/US2007/060584, filed on Jan. 16, 2007.
(60) Provisional application No. 60/949,768, filed on Jul. 13, 2007.
(60) Provisional application No. 60/758,886, filed on Jan. 13, 2006.

(51) Int. Cl. C07K 14/00; C07K 2/00

(52) U.S. Cl. 530/324; 530/350; 530/300

(57) ABSTRACT

The present invention relates to proteins and polypeptides as well as methods of using these proteins and polypeptides to: identify the location of an RXP-binding domain of Cx43CT, modulate a Cx43 gap junction channel, screen for compounds that modulate Cx43CT, and measure Cx43CT-binding affinity of a compound that binds to Cx43CT.

This application is a divisional application of U.S. patent application Ser. No. 12/172,355, filed Jul. 14, 2008, which claims the benefit of U.S. Provisional Patent Application No. 60/949,768, filed Jul. 13, 2007, and International Application No. PCT/US2007/060584, filed Jan. 16, 2007, and which claims the benefit of U.S. Provisional Patent Application No. 60/758,886, filed Jan. 13, 2006, each of which is hereby incorporated by reference in its entirety.

This invention was made with government support under grants GM57691, HL080602, GM072631, and HL39707 awarded by the National Institutes of Health. The government has certain rights in this invention.

### FIELD OF THE INVENTION

The present invention relates to the peptide-based regulation of gap junctions.

### BACKGROUND OF THE INVENTION

Connexins are integral membrane proteins that oligomerize to form intercellular channels called gap junctions. The most abundant gap junction protein in a number of mammalian systems is connexin43 (“Cx43”).

Gap junction channels are responsible for direct cell-to-cell communication. These channels are dynamic pores that are regulated in response to changes in the cellular environment and by protein interactions. In the heart, gap junction channels are a critical mechanism for the passage of electrical impulses (Lerner et al., “Accelerated Onset and Increased Incidence of Ventricular Arrhythmias Induced by Ischemia in Cx43-deficient Mice,” Circ. 101(5):547-552 (2000); Gutstein et al., “Conditional Gene Targeting of Connexin43: Exploring the Consequences of Gap Junction Remodeling in the Heart,” Cell Cornrnun. Adhes. 8(4-6):345-348 30 (2001); Vaidya et al., “Null Mutation of Connexin43 Causes Slow Propagation of Ventricular Activation in the Late Stages of Mouse Embryonic Development,” Circ. Res. 88(11): 1196-1202 (2001)). Each channel is composed of two identical hexameric structures or connexons that dock across an extracellular space. The result is a permeable pore that is dynamically regulated. The individual subunit of the connexon is the molecule connexin. This molecule resides in the membrane, with its N-terminal (“NT”), cytoplasmic loop (“CL”) and C-terminal (“CT”) domains in the cytoplasmic space, as illustrated in FIG. 1. In addition, there are four transmembrane domains and two extracellular domains that are involved in the docking to the opposing connexon. There are at least 20 different connexin isotypes in the mouse genome and 21 in the human genome (Willecke et al., “Structural and Functional Diversity of Connexin Genes in the Mouse and Human Genome,” Biol. Chem. 383(5):725-737 (2002)). The most abundant connexin isotype in the heart, brain and other tissues is the 43 KDa protein, Cx43.

Gap junctions allow the passage of ions and small molecules between cells and are regulated by a variety of chemical interactions between the connexin molecule and the microenvironment. As such, gap junctions act as active filters to control the passage of intercellular messages to modulate function.

Previous work has suggested that regulation of Cx43 channels results from the association of the CT domain, acting as a gating particle, and a separate region of the connexin molecule acting as a receptor for the gating particle (Duffy et al., “pH-Dependent Intramolecular Binding and Structure Involving Cx43 Cytoplasmic Domains,” J. Biol. Chem. 277(39):36706-36714 (2002); Moreno et al., “Role of the Carboxyl Terminal of Connexin43 in Transjunctional Fast Voltage Gating,” Circ. Res. 90(4):450-457 (2002), 92(1):e30 (2003) (erratum)). Additional studies have shown that this intra-molecular interaction can be modulated by other inter-molecular interactions in the microenvironment of the gap junction plaque (Morley et al., “Intramolecular Interactions Mediate pH Regulation of Connexin43 Channels,” Biophys. J. 70(3):1294-1302 (1996)). Thus, the emerging picture of a Cx43 gap junction plaque is that of a macromolecular complex where proteins act in concert to modulate intercellular communication. At the center of these interactions is the CT domain, which acts as a substrate for a number of kinases (Duffy et al., “Regulation of Connexin43 Protein Complexes by Intracellular Acidification,” Circ. Res. 94(2):215-222 (2004); Giepmans et al., “Interaction of c-Src with gap junction protein connexin-43. Role in the Regulation of Cell-cell Communication,” J. Biol. Chem. 276(11):8544-8549 (2001); Kanemitsu et al., “Tyrosine Phosphorylation of Connexin 43 by v-Src is Mediated by SH2 and SH3 Domain Interactions,” J. Biol. Chem. 272(36):22824-22831 (1997); Lampe et al., “Phosphorylation of Connexin43 on Serine-368 by Protein Kinase C Regulates Gap Junctional Communication,” J. Cell Biol. 149(7):1503-1512 (2000); Lau et al., “Regulation of Connexin43 Function by Activated Tyrosine Protein Kinases,” J. Bioenerg. Biomembr. 28(4):359-368 (1996); Shin et al., “The Regulatory Role of the C-Terminal Domain of Connexin43,” Cell Commun. Adhes. 8(4-6):271-275 (2001); TenBroek et al., “Ser364 of Connexin43 and the Upregulation of Gap Junction Assembly by cAMP,” J. Cell Biol. 155(7):1307-1318 (2001)), a ligand for noncatalytic proteins (Giepmans & Moolenaar, “The Gap Junction Protein Connexin43 Interacts with the Second PDZ Domain of the Zona Occludens-1 Protein,” Curr. Biol. 8(16):931-934 (1998); Giepmans et al., “Connexin-43 Interactions with ZO-1 and Alpha- and Beta-tubulin,” Cell Commun. Adhes. 8(4-6):219-223 (2001); Toyofuku et al., “Direct Association of the Gap Junction Protein Connexin-43 with ZO-1 in Cardiac Myocytes,” J. Biol. Chem. 273(21):12725-12731 (1998); Toyofuku et al., “c-Src Regulates the Interaction between Connexin-43 and ZO-1 in Cardiac Myocytes,” J. Biol. Chem. 276(3):1780-1788 (2000); Zhou et al., “Dissection of the Molecular Basis of pp 60(v-Src) Induced Gating of Connexin 43 Gap Junction Channels,” J. Cell Biol. 144(5):1033-1045 (1999); Ai et al., “Wnt-1 Regulation of Connexin43 in Cardiac Myocytes,” J. Clin. Invest. 105(2):161-171 (2000); Xu et al., “N-Cadherin and Cx43α1 Gap Junctions Modulates Mouse Neural Crest Cell Motility Via Distinct Pathways,” Cell Adhes. Commun. 8(4-6):321-324 (2001); Schubert et al., “Connexin Family Members Target to Lipid Raft Domains and Interact with Caveolin-1,” Biochem. 41(18):5754-5764 (2002)), and a gating particle to modify coupling between cells (Duffy et al., “pH-Dependent Intramolecular Binding and Structure Involving Cx43 Cytoplasmic Domains,” J. Biol. Chem. 277(39):36706-36714 (2002); Moreno et al., “Role of the Carboxyl Terminal of Connexin43 in Transjunctional Fast Voltage Gating,” Circ. Res. 90(4):450-457 (2002), 92(1):e30 (2003) (erratum); Morley et al., “Intramolecular Interactions Mediate pH Regulation of Connexin43 Channels,” Biophys. J. 70(3):1294-1302 (1996); Anumonwo et al., “The Carboxyl Terminal Domain Regulates the Unitary Conductance and Voltage Dependence of Connexin40 Gap Junction Channels,” Circ. Res. 88(7):666-673 (2001)).

The pharmacology of gap junctions has been reviewed by Srinivas et al. (Srinivas et al., “Prospects for Pharmacological Targeting of Gap Junction Channels,” in CARDIAC ELECTROPHYSIOLOGY: FROM CELL TO BEDSIDE 158-167 (Douglas Zipes & Jose Jalife eds., 4th ed. 2004)). There are few specific drugs for the modulation of gap junction function. Long chain alkanols (heptanol and octanol) have long been used as uncoupling agents. The mechanism of action is unknown although membrane fluidity is thought to be the primary target. General anesthetics have also been shown to uncouple gap junctions (Burt & Spray, “Volatile Anesthetics Block Intercellular Communication between Neonatal Rat Myocardial Cells,” Circ. Res. 65(3):829-837 (1989)), but, again, the mechanism is unknown and not specific to gap junction proteins. Some agents such as butanedione monoxime (Herve & Sarrouilhe, “Modulation of Junctional Communication by Phosphorylation: Protein Phosphatases, the Missing Link in the Chain,” Biol. Cell 94(7-8):423-432 (2002)) are thought to modify phosphorylation of gap junctions leading to uncoupling. Flufenamic acid has been shown to be an effective inhibitor of gap junctions but the mechanism is thought to be indirect and not specific to any connexin protein (Srinivas & Spray, “Closure of Gap Junction Channels by Arylaminobenzoates,” Mol. Pharmacol. 63(6):1389-1397 (2003)). Perhaps among the more specific inhibitors are the glycyrrhetinic acids (Davidson et al., “Reversible Inhibition of Intercellular Junctional Communication by Glycyrrhetinic Acid,” Biochem. & Biophys. Res. Commun. 134(1):29-36 (1986); Goldberg et al., “Evidence that Disruption of Connexon Particle Arrangements in Gap Junction Plaques is Associated with Inhibition of Gap Junctional Communication by a Glycyrrhetinic Acid Derivative,” Experimental Cell Res. 222(1):48-53 (1996)), which may also alter phosphorylation (Guan et al., “Gap junction Disassembly and Connexin 43 Dephosphorylation Induced by 18 beta-Glycyrrhetinic acid,” Mol. Carcinog. 16(3):157-164 (1996)). The most significant shortcomings of glycyrrhetinic acids are the lack of specificity for different connexin proteins and the lack of a specific mechanism of action. Interestingly, quinine blocks a selective group of gap junction channels (Srinivas et al., “Quinine Blocks Specific Gap Junction Channel Subtypes,” Proc. Nat'l Acad. Sci. U.S.A 98(19):10942-10947 (2001)) but does not block gap junctions formed by the connexins found in the heart.

Peptides have been shown to alter the function of gap junction channels. Antiarrhythmic peptide 10 (“AAP10”) alters gap junctional communication (Muller et al., “Actions of the Antiarrhythmic Peptide AAP10 on Intercellular Coupling,” Naunyn Schmiedebergs Arch. Pharmacol. 356(1):76-82 (1997); Dhein et al., “Effects of the New Antiarrhythmic Peptide ZP123 on Epicardial Activation and Repolarization Pattern,” Cell Commun. Adhes. 10(4-6):371-378 (2003)) indirectly by interaction with a membrane receptor and possibly by a PKC dependent mechanism (Dhein et al., “Protein Kinase Cα Mediates the Effect of Antiarrhythmic Peptide on Gap Junction Conductance,” Cell Commun. Adhes. 8(4-6):257-264 (2001)) which would lead to a variety of alterations in the target cell. Specific inhibition of gap junction formation has been demonstrated with the use of extracellular loop peptides (Kwak & Jongsma, “Selective Inhibition of Gap Junction Channel Activity by Synthetic Peptides,” J. Physiol. 516(3):679-685 (1999)). It is thought that these peptides inhibit gap junctions by preventing connexon docking in the extracellular gap. These effects are slow and the interaction requires high concentrations of peptide (Dahl et al., “Attempts to Define Functional Domains of Gap Junction Proteins with Synthetic Peptides,” Biophys. J. 67(5):1816-1822 (1994)).

What is needed is a strategy to use structural, binding and functional assays to develop a peptide-based approach to Cx43 regulation; and agents that can maintain gap junction channels in an open state.

The present invention is directed to overcoming these deficiencies in the art.

### SUMMARY OF THE INVENTION

A first aspect of the present invention relates to a protein or polypeptide comprising the formula A-[Wₘ-A]ₙ where each A is independently a peptide of formula XXXXRXPXXXX where each X is independently any amino acid, R is arginine, and P is proline. Each W is independently a linker, m is 1 or 2, and n is any number from 1 through 5.

A second aspect of the present invention relates to a protein or polypeptide that has an amino acid sequence consisting essentially of the formula XₙRXPXₘ where each X is independently any amino acid, R is arginine, P is proline, n is any number from zero to fifteen, m is any number from zero to fifteen, and the sum of n and m is any number from eight to fifteen.

A third aspect of the present invention relates to a protein or polypeptide comprising the formula A-Aₙ where each A is independently a peptide of formula XXXXRXPXXXX where each X is independently any amino acid, R is arginine, and P is proline; and n is any number from 1 through 5.

A fourth aspect of the present invention relates to a protein or polypeptide that has an amino acid sequence consisting essentially of the formula XₙRXPXₘ where each X is independently any amino acid, R is arginine, P is proline, n is any number from zero to seven, m is any number from zero to seven, and the sum of n and m is any number from zero to seven.

A fifth aspect of the present invention relates to a method of screening for compounds that modulate Cx43CT. This method involves contacting one or more candidate compounds with a polypeptide comprising an RXP-binding domain of Cx43CT, and identifying the candidate compounds that bind to the polypeptide as compounds that modulate Cx43CT.

A sixth aspect of the present invention relates to a method for measuring Cx43CT-binding affinity of a compound that binds to Cx43CT. This method involves contacting the compound with a polypeptide comprising an RXP-binding domain of Cx43CT, under conditions effective to permit binding and determining the dissociation constant for the interaction between the polypeptide and the compound.

A seventh aspect of the present invention relates to a method for identifying the location of an RXP-binding domain of Cx43CT. This method involves contacting a protein or polypeptide with Cx43CT under conditions effective to permit binding between the protein or polypeptide and an RXP-binding domain of Cx43CT, where the protein or polypeptide (i) has the formula A-[Wₘ-A]ₙ where each A is independently a peptide of formula XXXXRXPXXXX where each X is independently any amino acid, R is arginine, and P is proline; each W is independently a linker; m is 1 or 2; and n is any number from 1 through 5; (ii) consists essentially of the formula XₙRXPXₘ where each X is independently any amino acid, R is arginine, P is proline, n is any number from zero to fifteen, m is any number from zero to fifteen, and the sum of n and m is any number from eight to fifteen, (iii) has the formula A-Aₙ where each A is independently a peptide of formula XXXXRXPXXXX where each X is independently any amino acid, R is arginine, and P is proline; and n is any number from 1 through 5; or (iv) consists essentially of the formula XₙRXPXₘ where each X is independently any amino acid, R is arginine, P is proline, n is any number from zero to seven, m is any number from zero to seven, and the sum of n and m is any number from zero to seven; measuring resonance of one or more amino acids of the Cx43CT; and determining the location of any of the one or more amino acids whose resonance changes in the presence of the protein or polypeptide, where the location of the amino acids whose resonance changes in the presence of the protein or polypeptide indicates that the amino acid is located within an RXP-binding domain.

An eighth aspect of the present invention relates to a method for modulating a Cx43 gap junction channel. This method involves contacting the Cx43 gap junction channel with a compound that binds to an RXP-binding domain of the Cx43 gap junction channel under conditions effective to modulate the Cx43 gap junction channel.

The present invention supplies a feasible, peptide-based strategy to manipulate Cx43 regulation in native tissues. The proteins and polypeptides of the present invention can also be used as tools to characterize the specific role of gap junction regulation in health and disease.

Modulation of gap junction intercellular communication (“GJIC”) has potential as a pharmaceutical intervention in several diseases. It is well documented that closure of gap junctions during cardiac ischemia is a contributing factor in arrhythmias (Lerner et al., “The Role of Altered Intercellular Coupling in Arrhythmias Induced by Acute Myocardial Ischemia,” Cardiovasc. Res. 50(2):263-269 (2001); Lerner et al., “Accelerated Onset and Increased Incidence of Ventricular Arrhythmias Induced by Ischemia in Cx43-deficient Mice,” Circulation 101(5):547-552 (2000), which are hereby incorporated by reference in their entirety). It is therefore, possible that modulation of junctions such as with the present invention can alter the course of the arrhythmia.

It is also documented that tumor cells have lower connexin expression and GJIC than their normal counterparts (Naus, “Gap Junctions and Tumour Progression,” Can. J. Physiol. Pharmacol. 80(2):136-141 (2002); Mesnil, “Connexins and Cancer,” Biol. Cell 94(7-8):493-500 (2002); Ruch et al., “Defective Gap Junctional Intercellular Communication in Lung Cancer: Loss of an Important Mediator of Tissue Homeostasis and Phenotypic Regulation,” Exp. Lung Res. 27(3):231-243 (2001); Huang et al., “Reduced Connexin43 Expression in High-grade Human Brain Glioma Cells,” J. Surg. Oncol. 70(1):21-24 (1999)). It is also becoming clear that increasing gap junction intercellular communication may be a useful intervention in cancer (Krutovskikh et al., “Gap Junction Intercellular Communication Propagates Cell Death in Cancerous Cells,” Oncogene 21(13):1989-1999 (2002), 21(28):4471 (2002) (erratum); Salameh & Dhein, “Pharmacology of Gap Junctions. New Pharmacological Targets for Treatment of Arrhythmia, Seizure and Cancer?,” Biochim. Biophys. Acta 1719(1-2):36-58 (2005); Vine & Bertram, “Cancer Chemoprevention by Connexins,” Cancer Metastasis Rev. 21(3-4):199-216 (2002); Zhang et al., “The Gap Junction-independent Tumor-suppressing Effect of Connexin 43,” J. Biol. Chem. 278(45):44852-44856 (2003); Huang et al., “Connexin 43 (Cx43) Enhances Chemotherapy-induced Apoptosis in Human Glioblastoma Cells,” Int. J. Cancer 92(1):130-138 (2001)). Therefore, cancer treatment using the present invention is possible.

### BRIEF DESCRIPTION OF THE DRAWINGS

FIG. 1 is a schematic diagram showing the topology of a Cx43 subunit. “NT” is the amino terminal. “CL” is the cytoplasmic loop. “CT” is the carboxyl terminal domain. “L2” represents the portion of the CL from residues 119-144.

FIGS. 2A-D are graphs illustrating the balance of basic (“B”) and acidic (“A”) residues in peptides chosen at random from the RXP library (FIGS. 2A-C) or sequenced after selection for Cx43CT binding (FIG. 2D). The number of acidic residues (asp or glu) was subtracted from the number of basic residues (lys, arg or his) within the sequence. Thirty “doublets” were randomly assigned from the library (FIGS. 2A-C) and compared to 30 Cx43CT-bound peptides. A preponderance of 4 basic residues was found from the bound peptides (FIG. 2D).

FIG. 3A-B are Sensograms showing the results of SPR studies comparing the binding of RXP-4 and RXP-E to Cx43CT. FIG. 3A illustrates a Sensogram obtained from the binding of Cx43CT to RXP-4 (250 μM). The rapid and complete dissociation upon washout indicates weak intermolecular binding. FIG. 3B illustrates Sensograms showing concentration-dependent binding of RXP-E to Cx43CT. The increased amplitude and slower dissociation kinetics when compared to RXP-4 is apparent. Dissociation constant (K_{D}) was measured from the rate of association and dissociation, based on first rate order kinetics, and estimated to be 3.9 μM.

FIG. 4A-B are schematic diagrams showing resonance peaks corresponding to Cx43CT residues as recorded by an NMR ¹⁵N-HSQC protocol. Each insert depicts the contour of individual amino acids (noted on the top of the insert box) as recorded in the absence or the presence of either peptide RXP-4 (FIG. 4A) or RXP-E (FIG. 4B). For each panel, the top inserts are examples of amino acids whose position in the HSQC map was unaffected by the peptides; amino acids whose position in space was modified by the peptides are shown in the middle and bottom inserts. Both peptides caused a shift in the position of residues 376, 378 and 379, as well as residues 343-346.

FIG. 5A-B are graphs of junctional current (Iⱼ) traces obtained from Cx43-expressing N2a cell pairs before and during perfusion with 2 mM octanol (onset indicated by thick vertical arrow) (transjunctional voltage: 60 mV; pulse duration: 10 seconds; interpulse interval: 10 seconds). In FIG. 5A, patch pipettes were filled with normal internal pipette solution. In FIG. 5B, the pipette solution contained peptide RXP-E at a concentration of 0.1 mM. The presence of RXP-E in the patch pipette partially prevented octanol-induced uncoupling.

FIG. 6A-B are graphs of the time course of octanol-induced changes in coupling recorded from Cx43-expressing N2a cells. Experiments were conducted in the absence (“w/o RXPE;” N=7) or in the presence (“with RXPE;” N=10) of 0.1 mM RXP-E in the internal pipette solution. Time zero corresponds to the onset of octanol superfusion. “Uncoupling” refers to the complete loss of junctional current under the voltage clamp protocol illustrated in FIG. 5A-B and described in Example 12. FIG. 6A illustrates the percent of pairs that remained coupled at the end of each minute after onset of octanol. FIG. 6B shows the average junctional conductance (Gⱼ) as a function of time after onset of octanol. For each cell pair, Gⱼ was measured relative to the value recorded before octanol exposure. It is noted that RXP-E prevented complete uncoupling in all 10 pairs tested, though a reduction in Gⱼ was still apparent.

FIG. 7A-B are graphs showing the time course of octanol-induced uncoupling in M257-expressing N2a cells (M257 refers to a Cx43 construct truncated at amino acid 257, hence lacking most of the CT domain). Experiments were conducted in the absence (“w/o RXPE”; N=13) or in the presence (“with RXPE”; N=8) of 0.1 mM RXP-E in the internal pipette solution. Time zero corresponds to the onset of octanol superfusion. “Uncoupling” refers to the complete loss of junctional current under the voltage clamp protocol illustrated in FIG. 5A-B and described in Example 12. FIG. 7A shows the percent of pairs that remained coupled at the end of each minute after onset of octanol. FIG. 7B shows the average of junctional conductance (Gⱼ) as a function of time after onset of octanol. For each cell pair, Gⱼ was measured relative to the value recorded before octanol exposure. RXP-E failed to prevent uncoupling in cells expressing the truncated form of Cx43.

FIG. 8 is a graph illustrating the time course of changes in junctional conductance (Gⱼ) relative to control, as a function of time after patch break. Cells were filled with an internal pipette solution buffered at pH 6.2. Data depicted by open symbols and dashed line was obtained in the presence of RXP-E in the patch pipette (“with RXPE”; N=6). A separate set (“w/o RXPE”) was obtained from cell pairs recorded in the absence of RXP-E. The last set shows that a scrambled peptide has no effect on the low pHi induced closure of the channel. The data show that RXP-E exposure is associated with a partial prevention of acidification-induced uncoupling.

FIGS. 9A-C are a schematic diagram (FIG. 9A) and graphs (FIG. 9B-C) relating to single channel data obtained from Cx43-expressing N2a cells exposed to RXP-E (0.1 mM). FIG. 9A shows the original traces and all-point histograms (right of trace). Transjunctional voltage was +60 mV. Voltage pulses were held for 10 seconds. Traces were obtained from cell pairs showing a very low level of coupling (no uncoupling agents used). The pipette solution was pH 7.2 (upper trace) or pH 6.2 (middle and lower traces). FIG. 9B shows the frequency distribution histogram for unitary conductance (N=4, n=110) in the presence or absence of RXP-E. The histogram was best fit by a single Gaussian, centered at 100.5 pS. A peak corresponding to transitions in or out of the residual state is absent. FIG. 9C shows the open time of single channels in the presence or absence of RXP-E (no uncoupling agents used). An average of the open times measured yielded a value of 1.27 seconds (N=3, n=368). The latter contrasts with measurements obtained from Cx43 channels in the absence of RXP-E of 0.12 seconds (N=3, n=475).

FIG. 10A-B are junctional current recordings from N2a cells transfected with M257. Octanol was used to uncouple cells to demonstrate single channels in FIG. 10A. The recordings presented in FIG. 10B (representative of at least 5 traces) were obtained in the presence of RXP-E. The results indicate that the increase in the open time for wild-type Cx43 channels in the presence of RXP-E is not seen in M257 channels.

FIG. 11 is a western blot for Cx43. Glutathione beads fused to NL.RXPE (“GST-NL.RXPE”) or RXPE (“GST-RXPE”), and glutathione beads alone (“GST”), with (“±”) or without (“−”) exposure to heart lysate, were run on SDS-PAGE, transferred to a nitrocellulose membrane, and probed with an antibody for Cx43. Cx43 was not recovered when GST alone was used as bait. The two right lanes show that Cx43 was also recovered when peptide NL.RXPE was used as bait.

FIG. 12 is a graph illustrating the time course of changes in junctional conductance (Gⱼ) relative to control, as a function of time after patch break. The top line represents the normalized Gⱼ in the presence of peptide NL.RXPE (SEQ ID NO:11). The bottom line represents the normalized Gⱼ in the absence of NL.RXPE. Cells were treated with heptanol to reduce coupling and with a version of RXP-E without a linker component. The cell pairs with the peptide uncoupled at a much slower rate.

FIG. 13 is a graph of the percent of binding to Cx43 of various RXPE mutants relative to RXPE and a control (“GST”). Peptides 23, 25, 27, 29, 31, and 58 are mutants of RXPE in which the residues indicated above the bar have been replaced with alanine. Peptide 59 is a mutant of RXPE lacking the linker sequence.

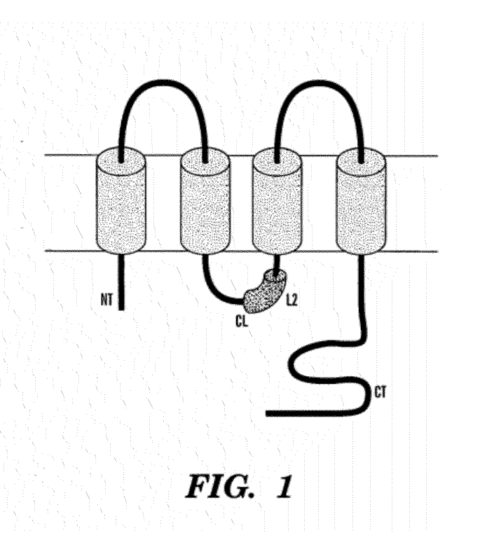

### DETAILED DESCRIPTION OF THE INVENTION

The present invention relates to a protein or polypeptide having the formula A-[Wₘ-A]ₙ where each A is independently a peptide of formula XXXXRXPXXXX where each X is independently any amino acid, R is arginine, and P is proline. Each W is independently a linker or absent, m is 1 or 2, and n is any number from 1 through 5.

Human Cx43 is a 382 aa protein that has the following amino acid sequence:

| (SEQ ID NO: 1) | |
|---|---|
| 1 | MGDWSALGKLLDKVQAYSTAGGKVWLSVLFIFRILLLGTAVESAWG |
| 47 | DEQSAFRCNTQQPGCENVCYDKSFPISHVRFWVLQIIFVSVPTLLYLAH |
| 96 | VFYVMRKEEKLNKKEEELKVAQTDGVNVDMHLKQIEIKKFKYGIEEH |

| | |
|---|---|
| 143 | GKVKMRGGLLRTYIISILFKSIFEVAFLLIQWYIYGFSLSAVYTCKRDPC |

| | |
|---|---|
| 193 | PHQVDCFLSRPTEKTIFIIFMLVVSLVSLALNIIELFYVFFKGVKDRVKG |
| 243 | KSDPYHATSGALSPAKDCGSQKYAYFNGCSSPTAPLSPMSPPGYKLVT |
| 291 | GDRNNSSCRNYNKQASEQNWANYSAEQNRMGQAGSTISNSHAQPFD |
| 337 | FPDDNQNSKKLAAGHELQPLAIVDQRPSSRASSRASSRPRPDDLEI |

(GenBank Accession No. NP 000156). As a key player in the regulation of gap junctions, the carboxy-terminal (“CT”; residues 255-382 of SEQ ID NO:1) presents itself as a potential target of chemical (Duffy et al., “pH-Dependent Intramolecular Binding and Structure Involving Cx43 Cytoplasmic Domains,” J. Biol. Chem. 277(39):36706-36714 (2002); Morley et al., “Intramolecular Interactions Mediate pH Regulation of Connexin43 Channels,” Biophys. J. 70(3):1294-1302 (1996); Homma et al., “A Particle-receptor Model for the Insulin-induced Closure of Connexin43 Channels,” Circ. Res. 83(1):27-32 (1998), which are hereby incorporated by reference in their entirety) or genetic manipulation intended to modify function (Moreno et al., “Role of the Carboxyl Terminal of Connexin43 in Transjunctional Fast Voltage Gating,” Circ. Res. 90:450-457 (2002); Morley et al., “Intramolecular Interactions Mediate pH Regulation of Connexin43 Channels,” Biophys. J. 70(3):1294-1302 (1996); Moorby & Patel, “Dual Functions for Connexins: Cx43 Regulates Growth Independently of Gap Junction Formation,” Exp. Cell Res. 271(2):238-248 (2001); Maass et al., “Defective Epidermal Barrier in Neonatal Mice Lacking the C-Terminal Region of Connexin43,” Mol. Biol. Cell 15(10):4597-4608 (2004), which are hereby incorporated by reference in their entirety). Here, it was sought to disrupt the regulation of Cx43 channels by chemical means. The rationale was based on the knowledge that Cx43CT is capable of interacting with other proteins. It was reasoned that this “stickiness” of Cx43CT can be used to “adhere” peptidic sequences to it. It was further thought that the interaction of Cx43CT with small peptides can modify the behavior of the gap junction channel. This rationale was supported by previous work showing that small peptides can modify both the chemical (Duffy et al., “pH-Dependent Intramolecular Binding and Structure Involving Cx43 Cytoplasmic Domains,” J. Biol. Chem. 277(39):36706-36714 (2002); Calero et al., “A 17mer Peptide Interferes with Acidification-induced Uncoupling of Connexin43,” Circ. Res. 82(9):929-935 (1998), which are hereby incorporated by reference in their entirety) and voltage gating (Seki et al., “Modifications in the Biophysical Properties of Connexin43 Channels by a Peptide of the Cytoplasmic Loop Region,” Circ. Res. 95(4):e22-e28 (2004), which is hereby incorporated by reference in its entirety) behavior of Cx43. In the present invention, a high throughput phage display screening was utilized to find peptidic sequences able to bind to Cx43CT. Further analysis using a combination of Surface Plasmon Resonance, Nuclear Magnetic Resonance and dual patch clamp led to the identification of a specific peptide that binds to Cx43CT with high affinity, affects residues 343-346 and 375-379 of Cx43 and partially prevents octanol-induced and acidification-induced uncoupling. These results support the feasibility of a peptide-based strategy to manipulate Cx43 regulation in native tissues. These peptides can be used as tools to characterize the specific role of gap junction regulation in health and disease (Shibayama et al., “Identification of a Novel Peptide that Interferes with the Chemical Regulation of Connexin-43,” Circ. Res. 98:1365-72 (2006), which is hereby incorporated by reference in its entirety).

The proteins and polypeptides of the present invention may be produced or isolated using methods known in the art. Suitable production methods include, for example, recombinant genetic engineering, chemical synthesis, and cell-free translation.

For example, a nucleic acid molecule encoding a polypeptide or protein of the present invention can be introduced into an expression system of choice using conventional recombinant technology. Generally, the nucleic acid molecule is incorporated into a vector-expression system of choice to prepare a nucleic acid construct using standard cloning procedures known in the art, such as those described in JOSEPH SAMBROOK & DAVID W. RUSSELL, MOLECULAR CLONING: A LABORATORY MANUAL (3d ed. 2001), which is hereby incorporated by reference in its entirety.

The nucleic acid molecule may be inserted into a vector in the sense (i.e., 5′→3′) direction, such that the open reading frame is properly oriented for the expression of the encoded protein under the control of a promoter of choice. Single or multiple nucleic acids may be ligated into an appropriate vector in this way, under the control of a suitable promoter, to prepare a nucleic acid construct for expressing a protein or polypeptide of the present invention. The vector should also contain the necessary elements for the transcription and translation of the inserted protein- or polypeptide-coding sequences.

Once the isolated nucleic acid molecule encoding the protein or polypeptide has been cloned into an expression system, it is ready to be incorporated into a host cell. Recombinant molecules can be introduced into cells via transformation, particularly transduction, conjugation, lipofection, protoplast fusion, mobilization, particle bombardment, electroporation (Neumann et al., “Gene Transfer into Mouse Lyoma Cells by Electroporation in High Electric Fields,” EMBO J. 1(7):841-845 (1982); Wong et al., “Electric Field Mediated Gene Transfer,” Biochem Biophys Res Commun 107(2):584-587 (1982); Potter et al., “Enhancer-dependent Expression of Human Kappa Immunoglobulin Genes Introduced into Mouse pre-B Lymphocytes by Electroporation,” Proc. Natl. Acad. Sci. USA 81(22):7161-7165 (1984), which are hereby incorporated by reference in their entirety), polyethylene glycol-mediated DNA uptake (JOSEPH SAMBROOK & DAVID W. RUSSELL, MOLECULAR CLONING: A LABORATORY MANUAL cp. 16 (2d ed. 1989), which is hereby incorporated by reference in its entirety), or fusion of protoplasts with other entities (e.g., minicells, cells, lysosomes, or other fusible lipid-surfaced bodies that contain the chimeric gene) (Fraley et al., “Liposome-mediated Delivery of Tobacco Mosaic Virus RNA into Tobacco Protoplasts: A Sensitive Assay for Monitoring Liposome-protoplast Interactions,” Proc. Natl. Acad. Sci. USA, 79(6):1859-1863 (1982), which is hereby incorporated by reference in its entirety). The nucleic acid molecules are cloned into the host cell using standard cloning procedures known in the art, as described in JOSEPH SAMBROOK & DAVID W. RUSSELL, MOLECULAR CLONING: A LABORATORY MANUAL (2d ed. 1989), which is hereby incorporated by reference in its entirety. Suitable hosts include, but are not limited to, bacteria, virus, yeast, fungi, mammalian cells, insect cells, plant cells, and the like.

The host cell is then cultured in a suitable medium, and under conditions suitable for expression of the protein or polypeptide of interest. After cultivation, the cell is disrupted by physical or chemical means, and the protein or polypeptide purified from the resultant crude extract. Alternatively, cultivation may include conditions in which the protein or polypeptide is secreted into the growth medium of the recombinant host cell, and the protein or polypeptide is isolated from the growth medium. Alternative methods may be used as suitable.

The proteins and polypeptides of the present invention can also be synthesized in a cell-free protein synthesis system. The above expression vector DNA is transcribed in vitro, and the resultant mRNA is added to a cell-free translation system to synthesize the protein. Specifically, the cell-free translation system is prepared from an extract of a eukaryotic cell or a bacterial cell, or a portion thereof. Such cell-free translation systems include those prepared from a rabbit reticulocyte, from a wheat germ, and from E. coli S30 extract.

Chemical synthesis can also be used to make suitable proteins or polypeptides. Such a synthesis is carried out using known amino acid sequences for the proteins and polypeptides of the present invention. These proteins and polypeptides can then be separated by conventional procedures (e.g., chromatography, SDS-PAGE) and used in the methods of the present invention. Suitable synthesis procedures include that described in Example 18.

The proteins and polypeptides of the present invention may be purified by methods that will be apparent to one of skill in the art.

Mutations or variants of the above polypeptides or proteins are encompassed by the present invention. Variants may be modified by, for example, the deletion or addition of amino acids that have minimal influence on the properties and secondary structure of the desired protein or polypeptide. For example, a protein or polypeptide of the present invention may be conjugated to a signal (or leader) sequence at the N-terminal end of the protein or polypeptide that co-translationally or post-translationally directs transfer of the protein or polypeptide. The protein or polypeptide may also be conjugated to another sequence for ease of synthesis, purification, or identification of the protein or polypeptide.

The linker in the protein or polypeptide according to this aspect of the present invention is preferably about 1 to about 15 amino acids long, more preferably about 1 to about 13 amino acids long, and most preferably about 8 to about 13 amino acids long. Suitable linkers include the formula Gly-Gly-Gly-Ser (SEQ ID NO:2), AHARVPFYSHS (SEQ ID NO:3), AETHARVPFYSHS (SEQ ID NO:4), and AVPFYSHS (SEQ ID NO:5). Alternatively, the linker is absent, i.e., the protein or polypeptide has the formula A-Aₙ.

In embodiments where m is 2, Wₘ may have a sequence of formula W₁-W₂, where W₁ and W₂ may be the same linker or different linkers. In one embodiment, W₁ is Gly-Gly-Gly-Ser (SEQ ID NO:2) and W₂ is AHARVPFYSHS (SEQ ID NO:3), AETHARVPFYSHS (SEQ ID NO:4), or AVPFYSHS (SEQ ID NO:5).

Preferably, n is any number from 1 through 4, most preferably 1 or 2. When n is any number greater than 1, W may be the same linker, different linkers, or combinations of the same linker and different linkers.

Exemplary proteins or polypeptides according to this aspect of the present invention include those having an amino acid sequence of

(SEQ ID NO: 6)
DVPGRDPGYIKGGGSAHARVPFYSHSLNRNRKPSLYQ,
(SEQ ID NO: 7)
EIQPRSPLMFSGGGSAHARVPFYSHSAKEARWPRAHR,
(SEQ ID NO: 8)
GIAAREPNSHDGGGSAHARVPFYSHSRDLWRKPAKSL,
(SEQ ID NO: 9)
WEEPRRPFTMSGGGSAETHARVPFYSHSPMRHRLPGVHL,
(SEQ ID NO: 10)
SDDLRSPQLHNGGGSAVPFYSHSHMVRRKPRNPR,
or
(SEQ ID NO: 11)
SDDLRSPQLHNHMVRRKPRNPR.

Preferably, the protein or polypeptide has at least 4 more basic amino acid residues than acidic amino acid residues.

The present invention also relates to a protein or polypeptide that has an amino acid sequence consisting essentially of the formula XₙRXPXₘ where each X is independently any amino acid, R is arginine, P is proline, and (i) n is any number from zero to fifteen, m is any number from zero to fifteen, and the sum of n and m is any number from eight to fifteen, or (ii) n is any number from zero to seven, m is any number from zero to seven, and the sum of n and m is any number from zero to seven.

In a preferred embodiment of the protein or polypeptide according to this aspect of the present invention, n is any number from one to eight. In another preferred embodiment, m is any number from one to eight. In another preferred embodiment, the sum of n and m is between 10 and 15. In another preferred embodiment, the sum of n and m is between 10 and 12. In another preferred embodiment, n is any number from one to eight, m is any number from one to eight, and the sum of n and m is between 10 and 12. In another preferred embodiment, the sum of n and m is between 1 and 7. In another preferred embodiment, the sum of n and m is between 1 and 5.

Exemplary proteins or polypeptides according to this aspect of the present invention include those having an amino acid sequence of

| | |
|---|---|
| GHLHLRVPTLKM, | (SEQ ID NO: 12) |
| EFIRSPHSVDWL, | (SEQ ID NO: 13) |
| SQSRNPPMPPPR, | (SEQ ID NO: 14) |
| RRPPYRVPPKLF, | (SEQ ID NO: 15) |
| SLYERHPASTYP, | (SEQ ID NO: 16) |
| HTVSRRPLPSSG, | (SEQ ID NO: 17) |
| RHTHGNLLRFPP, | (SEQ ID NO: 18) |
| RNNLNQTYPERR, | (SEQ ID NO: 19) |
| YSLLPVRPVALT, | (SEQ ID NO: 20) |
| RKPTQSLPTRLV, | (SEQ ID NO: 21) |
| TRRPHKMRSDPL, | (SEQ ID NO: 22) |
| TLTWHTKTPVRP, | (SEQ ID NO: 23) |
| SRQFLHSLDRLP, | (SEQ ID NO: 24) |
| HLHHHLDHRPHR, | (SEQ ID NO: 25) |
| QTPYQARLPAVA, | (SEQ ID NO: 26) |
| WHPHRHHHLQWD, | (SEQ ID NO: 27) |
| RRKPRRKP, | (SEQ ID NO: 28) |
| RNPRNP, | (SEQ ID NO: 29) |
| RRKP, | (SEQ ID NO: 30) |
| RRNP, | (SEO ID NO: 31) |
| or | |
| RNP. | |

Another aspect of the present invention relates to a method of screening for compounds that modulate Cx43CT. This method involves contacting one or more candidate compounds with a polypeptide comprising an RXP-binding domain of Cx43CT, and identifying the candidate compounds that bind to the polypeptide as compounds that modulate Cx43CT.

The RXP-binding domain according to this and all aspects of the present invention preferably includes amino acid residues 343-346 of SEQ ID NO:1, amino acid residues 375-379 of SEQ ID NO:1, or combinations thereof.

In a preferred embodiment, the method screens for candidate compounds that exhibit pH-dependent binding to Cx43CT. In this aspect of the present invention, a polypeptide comprising an RXP-binding domain of Cx43CT is contacted with a candidate compound that binds to the polypeptide, at a first pH under conditions effective to permit binding, and binding between the polypeptide and the compound is detected. In a separate experiment, the candidate compound is contacted with the polypeptide at a second pH under substantially similar conditions and binding between the polypeptide and the candidate compound is detected. The binding levels measured at the first and second pHs are compared, where a difference in binding level indicates that the candidate compound exhibits pH-dependent binding to Cx43CT.

Binding between the polypeptide and a candidate compound may be detected by a variety of mechanisms. One useful test is the use of Surface Plasmon Resonance (SPR) (Duffy et al., “Functional Demonstration of Connexin-protein Binding Using Surface Plasmon Resonance,” Cell Adhes. Commun. 8(4-6):225-229 (2001); Duffy et al., “pH-Dependent Intramolecular Binding and Structure Involving Cx43 Cytoplasmic Domains,” J. Biol. Chem. 277(39):36706-36714 (2002), which are hereby incorporated by reference in their entirety). In this system Cx43CT is bound to a matrix and the resonance of polarized residues is assessed. Binding of peptides to the Cx43CT alters that resonance and allows real-time determination of binding strength and kinetics.

Nuclear magnetic resonance (NMR) is also a technique that can determine whether two proteins interact, as well as determine where within the protein that binding takes place. This technique is described in detail below, and in Sorgen et al., “Sequence-specific Resonance Assignment of the Carboxyl Terminal Domain of Connexin43,” J. Biomol. NMR 23(3):245-246 (2002); Sorgen et al., “pH-Dependent Dimerization of the Carboxyl Terminal Domain of Cx43,” Biophys. J. 87(1):574-581 (2004); and Sorgen et al., “Structural Changes in the Carboxyl Terminus of the Gap Junction Protein Connexin43 Indicates Signaling between Binding Domains for c-Src and Zonula Occludens-1,” J. Biol. Chem. 279(2):54695-54701 (2004), which are hereby incorporated by reference in their entirety.

Another technique that may be used in the detection of protein binding is cross-linking (Sorgen et al., “pH-Dependent Dimerization of the Carboxyl Terminal Domain of Cx43,” Biophys. J. 87(1):574-581 (2004), which is hereby incorporated by reference in its entirety). This technique involves the association of two proteins followed by treatment with a chemical crosslinker to covalently link the two peptides. The proteins are resolved on a SDS-PAGE gel and a shift in size indicates protein crosslinking.

Another technique to measure the binding of two proteins is the use of an enzymatic binding system (Duffy et al., “Regulation of Connexin43 Protein Complexes by Intracellular Acidification,” Circ. Res. 94(2):215-222 (2004); Duffy et al., “Kinetics of Protein-protein Interactions of Connexins: Use of Enzyme Linked Sorbent Assays,” Cell Commun. Adhes. 10(4-6):207-210 (2003), which are hereby incorporated by reference in their entirety). In this system Cx43CT is immobilized and a potential binding peptide is added. The binding is detected by the activity of an enzyme linked to the peptide.

In another preferred embodiment, the method screens for candidate compounds that modulate the structure of Cx43CT. In this aspect of the present invention, a candidate compound is contacted with a polypeptide comprising an RXP-binding domain of Cx43CT under conditions effective to permit binding between the candidate compound and the polypeptide. Resonance of one or more amino acids of the polypeptide is measured. The resonance of the one or more amino acids of the polypeptide with and without the candidate compound is compared, where a difference in resonance of the one or more amino acids with and without the candidate compound indicates that the candidate compound modulates the structure of Cx43CT. The specific resonance peaks that correspond to each amino acid in the Cx43CT sequence have previously been assigned (Sorgen et al., “Sequence-specific Resonance Assignment of the Carboxyl Terminal Domain of Connexin43,” J. Biomol. NMR 23(3):245-246 (2002), which is hereby incorporated by reference in its entirety). Therefore, in this aspect of the present invention, the resonance of the one or more amino acids measured in the presence of the test compound can be compared to the previously-assigned resonance peaks. Alternatively, resonance of the one or more amino acids can be measured in the absence of the candidate compound to establish a reference for comparing resonance in the presence of the candidate compound.

Suitable methods for measuring resonance of amino acids include, for example, gradient-enhanced two-dimensional ¹H-¹⁵N HSQC experiments (Kay et al., “Pure Absorption Gradient Enhanced Heteronuclear Single Quantum Correlation Spectroscopy with Improved Sensitivity,” J. Am. Chem. Soc. 114:10663-10665 (1992), which is hereby incorporated by reference in its entirety).

In another preferred embodiment, the method screens for candidate compounds that modulate the activity of a Cx43 gap junction channel. In this aspect of the present invention, gap junction intercellular communication (“GJIC”) of a Cx43 gap junction channel is measured. In a separate experiment, a Cx43 gap junction channel is contacted with a candidate compound and the GJIC of the channel is measured under substantially similar conditions. The GJIC levels measured with and without the candidate compound are compared, where a difference in GJIC indicates that the candidate compound modulates the activity of the channel. Increase in GJIC indicates that the candidate compound increases the number of channels that form, increases the number of channels that open, and/or increases the duration of opening of a channel (i.e. the open state of the channel is maintained). Decrease in GJIC indicates that the candidate compound decreases the number of available channels, prevents opening of a channel, or reduces the duration of opening of a channel.

GJIC may be measured using methods known in the art. Suitable methods include measuring conductance of the gap junction channel, using, for example, dual-whole-cell voltage clamp assay (Seki et al., “Modifications in the Biophysical Properties of Connexin43 Channels by a Peptide of the Cytoplasmic Loop Region,” Circ. Res. 95(4):e22-e28 (2004), which is hereby incorporated by reference in its entirety).

Dye transfer assays may also be used to measure GJIC, which involve following the transfer of gap junction permeable dyes to neighboring cells. The basic transfer protocol involves the use of a mechanical scrape to open cells to a dye such as Lucifer yellow. This dye can pass through a gap junction and then be visualized in adjacent cells (el-Fouly et al., “Scrape-loading and Dye Transfer. A Rapid and Simple Technique to Study Gap Junctional Intercellular Communication,” Exp. Cell Res. 168(2):422-430 (1987), which is hereby incorporated by reference in its entirety). Alternatively, this dye may be microinjected into a single cell and transfer followed into neighboring cells (Lampe et al., “Phosphorylation of Connexin43 on Serine-368 by Protein Kinase C Regulates Gap Junctional Communication,” J. Cell Biol. 149(7):1503-1512 (2000)). In addition, flow cytometric analysis of dye transfer is an effective method to assess junctional activity (Goldberg et al., “A Pre-loading Method of Evaluating Gap Junctional Communication by Fluorescent Dye Transfer,” Biotechniques 18(3):490-497 (1995), 19(2):212 (1995) (erratum); Kavanagh et al., “Flow Cytometry and Scrape-loading/Dye Transfer as a Rapid Quantitative Measure of Intercellular Communication In Vitro,” Cancer Res. 47(22):6046-6051 (1987)).

In another preferred embodiment, the method screens for candidate compounds that modulate octanol-induced uncoupling of a gap junction channel. In this aspect of the present invention, two or more cells expressing one or more Cx43 gap junction channels are treated with octanol and the GJIC of the channel(s) is measured. In a separate experiment under substantially similar conditions, two or more cells expressing one or more Cx43 gap junction channels are treated with octanol, a candidate compound is contacted with the Cx43 gap junction channel(s), and the GJIC of the channel(s) is measured. The GJIC levels measured with and without the candidate compound are compared, where a difference in GJIC indicates that the candidate compound modulates octanol-induced uncoupling of a gap junction channel. Higher GJIC in the presence of the candidate compound indicates that the compound inhibits or reduces octanol-induced uncoupling of the channel. Lower GJIC in the presence of the candidate compound indicates that the compound enhances octanol-induced uncoupling of the channel.

In another preferred embodiment, the method screens for candidate compounds that modulate pH-dependent uncoupling of a gap junction channel. In this aspect of the present invention, two or more cells expressing one or more Cx43 gap junction channels are treated at a first pH and the GJIC of the channel(s) is measured. The pH of the extracellular fluid is changed to a second pH and the GJIC of the channel(s) is measured. The GJIC levels measured at the first and second pHs are compared, where a difference in GJIC indicates that there is pH-dependent uncoupling of a gap junction channel. In a separate experiment under substantially similar conditions, two or more cells expressing one or more Cx43 gap junction channels are treated at a first pH, a candidate compound is contacted with one or more of the channel(s), and the GJIC of the channel(s) is measured. The pH of the extracellular fluid is changed to a second pH, a candidate compound is contacted with one or more of the channel(s), the GJIC of the channel(s) is measured, and the GJIC levels measured at the first and second pHs are compared. The difference in GJIC measured in the first experiment is compared to the difference in GJIC measured in the second experiment, where change in the difference in GJIC indicates that the candidate compound modulates pH-dependent uncoupling of a gap junction channel. A smaller difference in GJIC measured in the second experiment (i.e. in the presence of the candidate compound) compared to the difference in GJIC measured in the first experiment (i.e. without the candidate compound) indicates that the candidate compound inhibits or reduces pH-dependent uncoupling of the channel. A larger difference in GJIC measured in the second experiment (i.e. in the presence of the candidate compound) compared to the difference in GJIC measured in the first experiment (i.e. without the candidate compound) indicates that the compound enhances pH-dependent uncoupling of the channel.

In another preferred embodiment, the method screens for candidate compounds that modulate uncoupling of a Cx43 gap junction channel in a CT-dependent manner. In this aspect of the present invention, two or more cells expressing one or more Cx43 gap junction channels are treated with octanol, a candidate compound is contacted with the Cx43 gap junction channel(s) and the GJIC of the channel(s) is measured. In a separate experiment under substantially similar conditions, two or more cells expressing one or more Cx43 gap junction channels that lack the CT domain are treated with octanol, the candidate compound is contacted with the Cx43 gap junction channel(s), and the GJIC of the channel(s) is measured. The GJIC levels measured with the Cx43 gap junction channel(s) and the channel(s) that lack the CT domain are compared, where higher or lower GJIC in the second experiment relative to the first experiment indicates that the candidate compound modulates uncoupling of a Cx43 gap junction channel in a CT-dependent manner. (Substantially the same GJIC indicates that the candidate compound either does not modulate uncoupling of the channel, or modulates uncoupling of the channel in a CT-independent manner. Whether the compound modulates octanol-induced uncoupling of the channel may be determined using the methods described above.)

In another preferred embodiment, the method screens for candidate compounds that stabilize a Cx43 gap junction channel in an open state. In this aspect of the present invention, mean open time of a Cx43 gap junction channel is measured. In a separate experiment, a candidate compound is exposed to a Cx43 gap junction channel and the mean open time of the channel is measured under substantially similar conditions. The mean open times measured with and without the candidate compound are compared, where a difference in mean open time indicates that the candidate compound stabilizes the open state of a Cx43 gap junction channel. An increase in mean open time indicates that the candidate compound stabilizes a Cx43 gap junction channel in an open state. A decrease in mean open time indicates that the candidate compound does not stabilize a Cx43 gap junction channel in an open state.

Mean open time of the gap junction channel may be measured, for example, as described in Examples 6 and 15 of the present application, and as described in Moreno et al., “Role of the Carboxyl Terminal of Connexin43 in Transjunctional Fast Voltage Gating,” Circ. Res. 90(4):450-457 (2002), 92(1):e30 (2003) (erratum), which is hereby incorporated by reference in its entirety.

Another aspect of the present invention relates to a method for measuring Cx43CT-binding affinity of a compound that binds to Cx43CT. This method involves contacting the compound with a polypeptide comprising an RXP-binding domain of Cx43CT, under conditions effective to permit binding and determining the dissociation constant for the interaction between the polypeptide and the compound.

The Cx43CT-binding affinity of a compound that binds to Cx43CT may be measured, for example, using the methods described in Salamon et al., “Surface Plasmon Resonance Spectroscopy as a Tool for Investigating the Biochemical and Biophysical Properties of Membrane Protein Systems. II: Applications to Biological Systems,” Biochim. Biophys. Acta 1331(2):131-152 (1997); Duffy et al., “Functional Demonstration of Connexin-protein Binding Using Surface Plasmon Resonance,” Cell Adhes. Commun. 8(4-6):225-229 (2001); Lang et al., “Surface Plasmon Resonance as a Method to Study the Kinetics and Amplitude of Protein-protein Binding,” in PRACTICAL METHODS IN CARDIOVASCULAR RESEARCH 936-947 (Stefan Dhein, Friedrich Wilhelm Mohr & Mario Delmar eds., 2005); Duffy et al., “pH-Dependent Intramolecular Binding and Structure Involving Cx43 Cytoplasmic Domains,” J. Biol. Chem. 277(39):36706-36714 (2002); Sorgen et al., “Sequence-specific Resonance Assignment of the Carboxyl Terminal Domain of Connexin43,” J. Biomol. NMR 23(3):245-246 (2002); Sorgen et al., “pH-Dependent Dimerization of the Carboxyl Terminal Domain of Cx43,” Biophys. J. 87(1):574-581 (2004); Sorgen et al., “Structural Changes in the Carboxyl Terminus of the Gap Junction Protein Connexin43 Indicates Signaling between Binding Domains for c-Src and Zonula Occludens-1,” J. Biol. Chem. 279(2):54695-54701 (2004); Duffy et al., “Regulation of Connexin43 Protein Complexes by Intracellular Acidification,” Circ. Res. 94(2):215-222 (2004); and Duffy et al., “Kinetics of Protein-protein Interactions of Connexins: Use of Enzyme Linked Sorbent Assays,” Cell Commun. Adhes. 10(4-6):207-210 (2003), which are hereby incorporated by reference in their entirety.

Another aspect of the present invention relates to a method for identifying the location of an RXP-binding domain of Cx43CT. This method involves contacting a protein or polypeptide according to the present invention with Cx43CT under conditions effective to permit binding between the protein or polypeptide and an RXP-binding domain of Cx43CT. Resonance of one or more amino acids of the Cx43CT is measured, and the location of any of the one or more amino acids whose resonance changes in the presence of the protein or polypeptide is determined. The location of the amino acids whose resonance changes in the presence of the protein or polypeptide indicates that the amino acid is located within an RXP-binding domain. Resonance may be measured and compared as described above.

Suitable proteins and polypeptides according to this aspect of the present invention include (i) those having the formula A-[Wₘ-A]ₙ where each A is independently a peptide of formula XXXXRXPXXXX where each X is independently any amino acid, R is arginine, and P is proline; each W is independently a linker; m is 1 or 2; and n is any number from 1 through 5; (ii) those that have an amino acid sequence consisting essentially of the formula XₙRXPXₘ where each X is independently any amino acid, R is arginine, P is proline, n is any number from zero to fifteen, m is any number from zero to fifteen, and the sum of n and m is any number from eight to fifteen; (iii) those having the formula A-Aₙ where each A is independently a peptide of formula XXXXRXPXXXX where each X is independently any amino acid, R is arginine, and P is proline; and n is any number from 1 through 5; and (iv) those that have an amino acid sequence consisting essentially of the formula XₙRXPXₘ where each X is independently any amino acid, R is arginine, P is proline, n is any number from zero to seven, m is any number from zero to seven, and the sum of n and m is any number from zero to seven. Specific examples of such proteins and polypeptides are identified above.

Another aspect of the present invention relates to a method for modulating a Cx43 gap junction channel. This method involves contacting the Cx43 gap junction channel with a compound that binds to an RXP-binding domain of the Cx43 gap junction channel under conditions effective to modulate the Cx43 gap junction channel.

This aspect of the present invention may be carried out in vitro or in vivo.

When the method of the present invention is carried out in vivo, the compounds that bind to an RXP-binding domain of the Cx43 gap junction channel may be administered to a subject under conditions effective to contact the Cx43 gap junction channel with the compound. These compounds can be administered orally, parenterally, for example, intradermally, subcutaneously, intravenously, intramuscularly, intraperitoneally, by intranasal instillation, or by application to mucous membranes, such as that of the nose, throat, and bronchial tubes. They may be administered alone or with suitable pharmaceutical carriers, and can be in solid or liquid form, such as tablets, capsules, powders, solutions, suspensions, or emulsions.

The compounds may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These compounds may also be administered parenterally. Solutions or suspensions of these compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds may also be administered directly to the airways in the form of an aerosol. For use as aerosols, the compounds in solution or suspension may be packaged in a pressurized aerosol container together with suitable propellants, for example, hydrocarbon propellants like propane, butane, or isobutane with conventional adjuvants. The compounds also may be administered in a non-pressurized form such as in a nebulizer or atomizer.

The compounds may be administered directly to the targeted tissue. Additionally and/or alternatively, the compounds may be administered to a non-targeted area along with one or more tissue-specific agents that facilitate migration of the compounds to the targeted tissue.

This aspect of the present invention may be carried out, for example, to modulate the opening of the gap junction channel and/or to modulate the closing of the gap junction channel.

Opening of the gap junction channel may be modulated by contacting the channel with a compound that, for example, prevents opening of the channel, decreases the degree to which the channel opens, promotes opening of the channel, increases the degree to which the channel opens, or stabilizes the channel in an open state. Closing of the gap junction channel may be modulated by contacting the channel with a compound that, for example, prevents closing of the channel, decreases the degree to which the channel closes, promotes closing of the channel, increases the degree to which the channel closes, or stabilizes the channel in a closed state.

Suitable proteins and polypeptides according to this aspect of the present invention include, for example, (1) those having the formula A-[Wₙ-A]ₙ where each A is independently a peptide of formula XXXXRXPXXXX where each X is independently any amino acid, R is arginine, and P is proline; each W is independently a linker; m is 1 or 2; and n is any number from 1 through 5; (ii) those that have an amino acid sequence consisting essentially of the formula XₙRXPXₘ where each X is independently any amino acid, R is arginine, P is proline, n is any number from zero to fifteen, m is any number from zero to fifteen, and the sum of n and m is any number from eight to fifteen; (iii) those having the formula A-Aₙ where each A is independently a peptide of formula XXXXRXPXXXX where each X is independently any amino acid, R is arginine, and P is proline; and n is any number from 1 through 5; and (iv) those that have an amino acid sequence consisting essentially of the formula XₙRXPXₘ where each X is independently any amino acid, R is arginine, P is proline, n is any number from zero to seven, m is any number from zero to seven, and the sum of n and m is any number from zero to seven. Specific examples of such proteins and polypeptides are identified above.

Suitable compounds according to this aspect of the present invention include, for example, compounds that: exhibit pH-depending binding to Cx43CT; modulate structure of Cx43CT; modulate activity of the Cx43 gap junction channel; modulate octanol-induced uncoupling of the Cx43 gap junction channel; modulate pH-dependent uncoupling of the Cx43 gap junction channel; modulate CT-dependent uncoupling of the Cx43 gap junction channel; or stabilize the Cx43 gap junction channel in an open state. Compounds that have a combination of these effects are also contemplated. Suitable compounds may be identified according to the methods disclosed herein.

The present invention may be further illustrated by reference to the following examples.

### EXAMPLES

### Example 1

### Production of Recombinant Protein

Recombinant Cx43CT was produced as described in Duffy et al., “pH-Dependent Intramolecular Binding and Structure Involving Cx43 Cytoplasmic Domains,” J. Biol. Chem. 277(39):36706-36714 (2002), which is hereby incorporated by reference in its entirety. Briefly, cDNA derived from rat Cx43 was inserted into pGEX-6P-2 (Amersham) and expressed in E. coli (BL-21). The resultant GST-fusion protein was cleaved from the GST PreScission Protease® (Amersham Biosciences). The recombinant product after cleavage contained the sequence 255-382 of rCx43 preceded by four additional amino acids (GPLG) (SEQ ID NO: 40). Protein concentration was measured using the Bio-Rad DC Protein Assay. Protein purity was assessed by SDS-PAGE.

### Example 2

### Phage Display

A “library” of bacteriophage, displaying ˜55 copies of 2.7×10⁹ random 12-mer peptides (Ph.D.-12™ Phage display peptide library kit; New England BioLabs Inc.) was utilized. Wells in 24-well plates were coated with 15 μg of recombinant CT and blocked with 5 mg/ml BSA. After removing the unbound viruses by washing with Tris buffered saline containing 0.5% tween-20 (TBS-T), low affinity binders were eluted with a solution of TBS-containing Cx43CT at a concentration of 100 μg/ml. High affinity binders were recovered by overlaying the well with a culture of E. coli, to allow the tightly bound phage to infect the bacteria. This culture was amplified and the virus was precipitated with PEG. The amplified product was used for the subsequent round of panning All steps were conducted at pH 6.5 unless otherwise indicated.

After three rounds of selection and amplification, the phage recovered from the last round of binding were grown on a lawn of E. coli for plaque purification.

Each plaque, representing a single clone, was picked and amplified. The phage were isolated and analyzed by DNA sequencing. The peptide sequences displayed were deduced from these sequences.

### Example 3

### Biased Phage Display

The Ph.D. Peptide Display Cloning System (New England BioLabs Inc.) was used to create a biased phage display library of sequence XXXXRXPXXXX, where X is any amino acid flanking an arginine and a proline with an additional random residue at the center. The randomized peptides were followed by a Gly-Gly-Gly-Ser (SEQ ID NO:2) spacer. The library was cloned into M13 phage. These phage were analyzed for titer and sequence prior to screening for Cx43CT binding as described above.

### Example 4

### Surface Plasmon Resonance (SPR)

SPR is a spectroscopic method to determine binding amplitude and kinetics in real time (Salamon et al., “Surface Plasmon Resonance Spectroscopy as a Tool for Investigating the Biochemical and Biophysical Properties of Membrane Protein Systems. II: Applications to Biological Systems,” Biochim. Biophys. Acta 1331(2):131-152 (1997); Duffy et al., “Functional Demonstration of Connexin-protein Binding Using Surface Plasmon Resonance,” Cell Adhes. Commun. 8(4-6):225-229 (2001); Lang et al., “Surface Plasmon Resonance as a Method to Study the Kinetics and Amplitude of Protein-protein Binding,” in PRACTICAL METHODS IN CARDIOVASCULAR RESEARCH 936-947 (Stefan Dhein, Friedrich Wilhelm Mohr & Mario Delmar eds., 2005), which are hereby incorporated by reference in their entirety). Recombinant Cx43CT was covalently bound to a carboxylmethyl dextran matrix (Salamon et al., “Surface Plasmon Resonance Spectroscopy as a Tool for Investigating the Biochemical and Biophysical Properties of Membrane Protein Systems. II: Applications to Biological Systems,” Biochim. Biophys. Acta 1331(2):131-152 (1997), which is hereby incorporated by reference in its entirety). Specific peptides were presented and, when feasible, dissociation constants (K_{D}) were calculated from the time course of binding and unbinding of the ligand, using a 1:1 (Langmuir) association and dissociation kinetic model (Biacore software package). In both phases (association and dissociation), the first 5-8 seconds of recording were not included in the fit, to avoid artifacts resulting from peptide distribution within the flow cells (Lang et al., “Surface Plasmon Resonance as a Method to Study the Kinetics and Amplitude of Protein-protein Binding,” in PRACTICAL METHODS IN CARDIOVASCULAR RESEARCH 936-947 (Stefan Dhein, Friedrich Wilhelm Mohr & Mario Delmar eds., 2005), which is hereby incorporated by reference in its entirety).

### Example 5

### Nuclear Magnetic Resonance (NMR)

All NMR data were acquired on a Varian INOVA 600-MHz NMR spectrometer using a cryoprobe (MacUra & Ernst, “Elucidation of Cross Relaxation in Liquids by Two-dimensional NMR Spectroscopy,” Mol. Phys. 41:95-117 (1980), which is hereby incorporated by reference in its entirety); the sample temperature was maintained at 7° C. Gradient-enhanced two-dimensional ¹H-¹⁵N HSQC experiments (Kay et al., “Pure Absorption Gradient Enhanced Heteronuclear Single Quantum Correlation Spectroscopy with Improved Sensitivity,” J. Am. Chem. Soc. 114:10663-10665 (1992), which is hereby incorporated by reference in its entirety) were used to observe all backbone amide resonances in ¹⁵N-labeled Cx43CT. Data were acquired with 1024 complex points in t₂ and 128 complex points in t₁. Sweep widths were 10,000 Hz in the proton dimension and 2,500 Hz in the nitrogen dimension. The concentration of RXP peptide to Cx43CT was approximately 2.4 mM to 0.8 mM, respectively (3:1 ratio). All NMR data were processed using NMRPipe (Delaglio et al., “NMRPipe: A Multidimensional Spectral Processing System Based on UNIX Pipes,” J. Biomol. NMR 6(3):277-293 (1995), which is hereby incorporated by reference in its entirety) and analyzed using NMRView (Sorgen, “How to Solve a Protein Structure by Nuclear Magnetic Resonance—The Connexin43 Carboxyl Terminal Domain,” in PRACTICAL METHODS IN CARDIOVASCULAR RESEARCH 948-958 (Stefan Dhein, Friedrich Wilhelm Mohr & Mario Delmar eds., 2005), which is hereby incorporated by reference in its entirety).

### Example 6

### Electrophysiological Analysis

Experiments were conducted on N2a (Neuroblastoma) cells. Cx43 was expressed either in a lac-switch stable system (induced by 0.1-1.0 mM of IPTG (Zhong et al., “LacSwitch II Regulation of Connexin43 cDNA Expression Enables Gap junction Single-channel Analysis,” Biotechniques 34(5):1034-1034, 1041-1044, 1046 (2003), which is hereby incorporated by reference in its entirety)) or transiently using an IRES plasmid coding for eGFP (Seki et al., “Modifications in the Biophysical Properties of Connexin43 Channels by a Peptide of the Cytoplasmic Loop Region,” Circ. Res. 95(4):e22-e28 (2004); Seki et al., “Loss of Electrical Communication, but Not Plaque Formation, After Mutations in the Cytoplasmic Loop of Connexin43,” Heart Rhythm. 1(2):227-233 (2004), which are hereby incorporated by reference in their entirety). M257 (a mutant of Cx43 truncated at amino acid 257 (Morley et al., “Intramolecular Interactions Mediate pH Regulation of Connexin43 Channels,” Biophys. J. 70(3):1294-1302 (1996), which is hereby incorporated by reference in its entirety)) was transiently expressed in N2a cells also using an eGFP-containing IRES plasmid. Cells were placed on the stage of an inverted microscope equipped for epifluorescence (Nikon Diaphoto200, Filter: 520 to 560 nm). Junctional current (Iⱼ) was recorded from eGFP-positive cell pairs in a dual-whole-cell voltage clamp configuration (holding potentials: −40 mV; transjuctional voltage, Vⱼ, +60 MV; step duration, 10-30 seconds). Patch pipettes were filled with a cesium-containing solution (Seki et al., “Modifications in the Biophysical Properties of Connexin43 Channels by a Peptide of the Cytoplasmic Loop Region,” Circ. Res. 95(4):e22-e28 (2004); Seki et al., “Loss of Electrical Communication, but Not Plaque Formation, After Mutations in the Cytoplasmic Loop of Connexin43,” Heart Rhythm. 1(2):227-233 (2004), which are hereby incorporated by reference in their entirety). For the low pH experiments, HEPES was replaced by MES (10 mM). Pipette resistance was 4.0-6.0MΩ, Synthetic peptides were diluted in the pipette solution to a final concentration of 0.1 mM. During recording, cells were kept at room temperature in a cesium-containing solution (Seki et al., “Modifications in the Biophysical Properties of Connexin43 Channels by a Peptide of the Cytoplasmic Loop Region,” Circ. Res. 95(4):e22-e28 (2004); Seki et al., “Loss of Electrical Communication, but Not Plaque Formation, After Mutations in the Cytoplasmic Loop of Connexin43,” Heart Rhythm. 1(2):227-233 (2004), which are hereby incorporated by reference in their entirety). For some experiments, octanol (2.0 mM) was superfused during recording. Data acquisition and recording, and criteria for single channel detection were as reported in Seki et al., “Modifications in the Biophysical Properties of Connexin43 Channels by a Peptide of the Cytoplasmic Loop Region,” Circ. Res. 95(4):e22-e28 (2004) and Seki et al., “Loss of Electrical Communication, but Not Plaque Formation, After Mutations in the Cytoplasmic Loop of Connexin43,” Heart Rhythm. 1(2):227-233 (2004), which are hereby incorporated by reference in their entirety.

### Example 7

### Non-Biased Phage Display

Initial control experiments were conducted to standardize the phage display assay. The library was presented to purified streptavidin, a protein known to bind preferentially to peptides containing an HPQ consensus motif (Devlin et al., “Random Peptide Libraries: A Source of Specific Protein Binding Molecules,” Science 249(4967):404-406 (1990); Blasko et al., “Mechanistic Studies with Potent and Selective Inducible Nitric-oxide Synthase Dimerization Inhibitors,” J. Biol. Chem. 277(1):295-302 (2002), which are hereby incorporated by reference in their entirety). After three rounds of enrichment, 14 phage plaques were selected for sequencing. All 14 showed the preservation of the HPQ motif, indicating that the experimental conditions were adequate for appropriate peptide recognition by the target.

The library was presented to Cx43CT. After three rounds of selection and amplification, DNA of 156 phage plaques was purified and sequenced. Of the estimated 2.7×10⁹ different sequences in the library, 48 unique sequences were recovered. One particular sequence (PRPTMGNLPDVL) (SEQ ID NO:32) was recovered from 45 different plaques. This particular peptide showed strong homology (using the GAP routine of the Wisconsin package, GCG software) with a 10 amino acid segment (RATLLNVPDL) (SEQ ID NO:33) of the second PDZ domain of the tight junction protein, zonula ocludens-1 (ZO-1). Binding between the second PDZ domain of ZO-1 and Cx43CT in vivo and in vitro has been well documented (Giepmans et al., “Connexin-43 Interactions with ZO-1 and Alpha- and Beta-tubulin,” Cell Commun. Adhes. 8(4-6):219-223 (2001); Sorgen et al., “Structural Changes in the Carboxyl Terminus of the Gap Junction Protein Connexin43 Indicates Signaling between Binding Domains for c-Src and Zonula Occludens-1,” J. Biol. Chem. 279(2):54695-54701 (2004), which are hereby incorporated by reference in their entirety). The results therefore support the notion that the phage display method can be helpful at recognizing Cx43CT binding sequences.

### Example 8

### The “RXP” Motif and Biased Phage Display

Further analysis of the 12-mer peptides identified as described in Example 7 revealed that 16 out of 48 unique sequences shared a motif “RXP” (where X represents any amino acid). The motif was shown in 11 peptides in the N- to C-terminal orientation and in 5 peptides in the reverse direction. Specific sequences are presented in Table 1.

TABLE 1
Alignment of 16 RXP-containing sequences derived from the phage display
screen.

| | | |
|---|---|---|
| RXP1 | N - G H L H L R V P T L K M - C | (SEQ ID NO: 12) |
| RXP2 | N - E F I R S P H S V D W L - C | (SEQ ID NO: 13) |
| RXP3 | N - S Q S R N P P M P P P R - C | (SEQ ID NO: 14) |
| RXP4 | N - R R P P Y R V P P K L F - C | (SEQ ID NO: 15) |
| RXP5 | N - S L Y E R H P A S T Y P - C | (SEQ ID NO: 16) |
| RXP6 | N - H T V S R R P L P S S G - C | (SEQ ID NO: 17) |
| | N - R H T H G N L L R F P P - C | (SEQ ID NO: 18) |
| | C - R R E P Y T Q N L N N R - N | (SEQ ID NO: 19) |
| | C - T L A V P R V P L L S Y - N | (SEQ ID NO: 20) |
| | N - R K P T Q S L P T R L V - C | (SEQ ID NO: 21) |
| | N - T R R P H K M R S D P L - C | (SEQ ID NO: 22) |
| | C - P R V P T K T H W T L T - N | (SEQ ID NO: 23) |
| | N - S R Q F L H S L D R L P - C | (SEQ ID NO: 24) |
| | C - R H P R H D L H H H L H - N | (SEQ ID NO: 25) |
| | N - Q T P Y Q A R L P A V A - C | (SEQ ID NO: 26) |
| | C - D W Q L H H H R H P H W - N | (SEQ ID NO: 27) |

The RXP motif was not detected in phage that bound to streptavidin. The motif occurred in different positions within the 12-mer peptide, thus preventing proper alignments to determine the frequency of other amino acids at specific positions relative to the RXP. To overcome this limitation and to search for peptides with higher binding affinity (see Example 10), a biased phage display library was generated where the RXP motif was forced to the center of the sequence, flanked at each side by 4 randomized amino acids. Sixty clones containing an insert (out of an estimated 3-4×10⁴ total clones in the library) were chosen at random for sequencing prior to exposure of the library to Cx43CT. All inserts coded for an 11-mer peptide with the RXP motif in the appropriate location. In three independent runs, the same library was presented to Cx43CT. The binding step was carried out at pH 6.5 in two of the runs (run 1 and 2) and at pH 7.4 in one additional run (run 3). The total number of phage plaques containing a full coding insert that were recovered after the binding step were 120, 119 and 163 for runs 1, 2 and 3, respectively. The results showed that 89% of all sequences corresponded to “doublets,” that is, peptides where two RXP 11-mers had been inserted in tandem. These inserts may be consequent to incomplete restriction enzyme digestion as well as possible residual activity of the Klenow polymerase used in second strand production. The combination of these two factors would result in blunt ends that subsequently ligated. Analysis of the doublet sequences indicated that one or both of these processes occurred in each doublet. Furthermore, five specific peptides were recovered from all three runs and represented a large fraction of the repeats observed within the same run (see Table 2).

TABLE 2
Frequency of occurrence of doublet peptides in
biased phage display screening.

| | | | | | | Total |
|---|---|---|---|---|---|---|
| | | Total inserts | Total | Total Unique | Total | Unique |
| Run | pH | sequenced | Doublets | Doublets | Singlets | Singlets |
| 1 | 6.5 | 120 | 102 | 18 | 18 | 18 |
| 2 | 6.5 | 119 | 107 | 10 | 12 | 10 |
| 3 | 7.4 | 163 | 150 | 19 | 13 | 13 |
| Totals | | 402 | 359 | 47 | 43 | 41 |

Sequences of the five most commonly identified peptides and their frequency in each run are provided in Table 3.

TABLE 3
Most commonly identified peptides.

| | | Frequency | Frequency | Frequency | Total |
|---|---|---|---|---|---|
| | | Run 1 | Run 2 | Run 3 | Frequency |
| Code | Sequence | (%) | (%) | (%) | (%) |
| RXP-A | DVPGRDPGYIKGGGS(1)LNR | 20.8 | 64.7 | 36.2 | 40 |
| | NRKPSLYQ | | | | |
| | (SEQ ID NO: 6) | | | | |
| RXP-B | EIQPRSPLMFSGGGS(1)AKE | 6.7 | 2.5 | 13.5 | 8.2 |
| | ARWPRAHR | | | | |
| | (SEQ ID NO: 7) | | | | |
| RXP-C | GIAAREPNSHDGGGS(1)RDL | 12.5 | 11.8 | 19.6 | 15.2 |
| | WRKPAKSL | | | | |
| | (SEQ ID NO: 8) | | | | |
| RXP-D | WEEPRRPFTMSGGGS(2)PM | 16.7 | 0.8 | 1.8 | 6 |
| | RHRLPGVHL | | | | |
| | (SEQ ID NO: 9) | | | | |
| RXP-E | SDDLRSPQLHNGGGS(3)HMV | 0.8 | 4.2 | 3.7 | 3 |
| | RRKPRNPR | | | | |
| | (SEQ ID NO: 10) | | | | |

(1) = linker “AHARVPFYSHS” (SEQ ID NO: 3)
(2) = linker “AETHARVPFYSHS” (SEQ ID NO: 4)
(3) = linker “AVPFYSHS” (SEQ ID NO: 5)

The absence of doublets in the initial library prior to screening and their high frequency after the binding step strongly suggest that these peptides, though rare, were highly selected for Cx43CT binding.

All three runs described above were carried out with the same library. As a further control, a new library was generated and the entire screening procedure was repeated. An attempt was made to minimize the occurrence of doublets by increasing restriction enzyme digestion time as well as the temperature of heat inactivation of the Klenow fragment. Despite the modification in the protocol, the results were similar to those above. No doublets were found in the pre-bound library and yet multimers represented the highest occurrence of bound peptides. 19 inserts selected at random from the library prior to the binding step were screened, all of which corresponded to the coding sequence of an 11-mer peptide with the RXP motif at the center. In contrast, 43 out of a total of 44 phage recovered after the binding step and containing an insert corresponded to multimers of RXP. The frequency of occurrence of the different multimers is presented in Table 4.

TABLE 4
Summary of sequences obtained from modified phage display procedure.

| | | | | Total Frequency |
|---|---|---|---|---|
| | | Total | Frequency (%) | (%) |
| | Sequences | 44 | — | — |
| | Screened | | | |
| Multimers | Doublets | 30 | 68.2 | 97.7 |
| | Triplets | 12 | 27.3 | |
| | Quadruplets | 1 | 2.3 | |
| Singlets | Singlet | 1 | 2.3 | 2.3 |

These results confirm that doublets are highly selected during the binding step.

### Example 9

### Frequency of Basic Residues

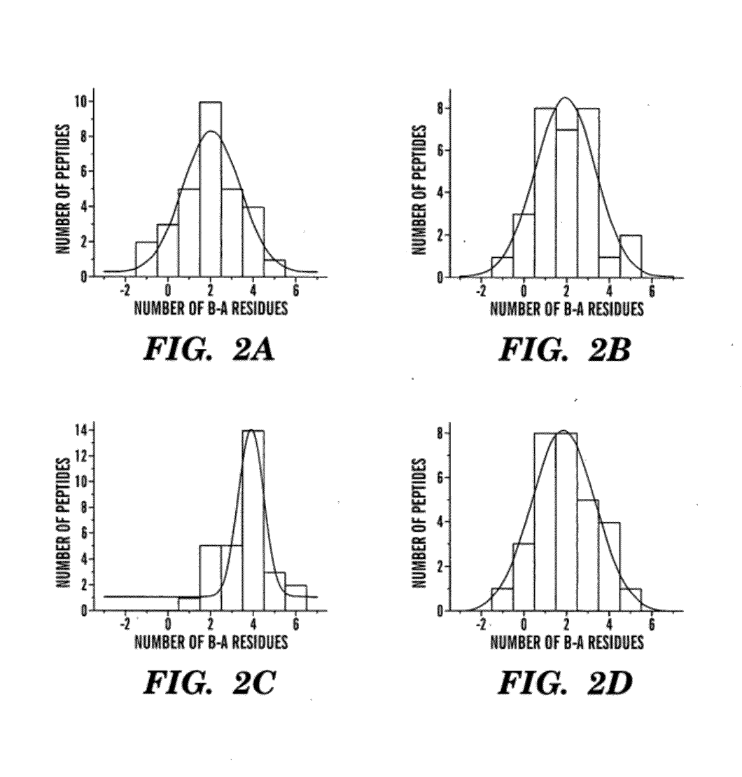

Although a clear preference for specific amino acid groups in a given position relative to the RXP core was not detected, a preponderance of basic residues in sequences recovered from all screenings was observed. To determine whether Cx43CT selected for peptides with a higher number of basic versus acidic residues, the frequency of each group of amino acids (and the balance between them) was determined in all doublets recovered. To simplify the analysis, the linker sequence in the doublets was not taken into account (see Example 15). As a control, the sequences recovered from the pre-bound library were used. As all sequences detected from the control library corresponded to single 11-mer peptides, they were randomly paired, forming theoretical “22-mer” peptides that compared in length to the doublets. To confirm that no bias occurred in the pairing of the control peptides, the amino acid frequency in “pairs” conformed in three different random assignments was characterized. As shown in FIGS. 2A-D, the balance of basic versus acidic residues in the library was well described by Gaussian functions. The numbers in the abscissas correspond to the algebraic sum of the acidic and basic residues in an individual peptide. The arginine residue in the center of the sequence (part of the “RXP” motif) was included in the count. FIGS. 2A-C show the frequency histograms obtained from the randomized pairs of control peptides. Each basic residue (H, K or R) was assigned a value of +1 and each acidic residue (D or E) was assigned a value of −1. Sixty (60) control peptides (thirty “pairs”) were used and compared to the 30 doublets recovered from all screenings. Each unique doublet was counted as one, regardless of the frequency with which it appeared after Cx43CT binding. As shown in FIGS. 2A-C, Gaussians were centered near 2.0 for the control peptides. In other words, regardless of the randomization, the control peptides had a balance of two basic residues (consistent with the fact that the library was biased to express one arginine in each 11-mer). In contrast, the frequency histogram obtained from the doublets peptides that bound Cx43CT had an abundance of 4 basic over acidic residues as shown in FIG. 2D. These results indicate that Cx43CT preferentially bound peptides with a higher number of basic residues, perhaps consequent to the positive balance of charge within the peptide.

### Example 10

### In Vitro Binding Detected by Surface Plasmon Resonance (SPR)

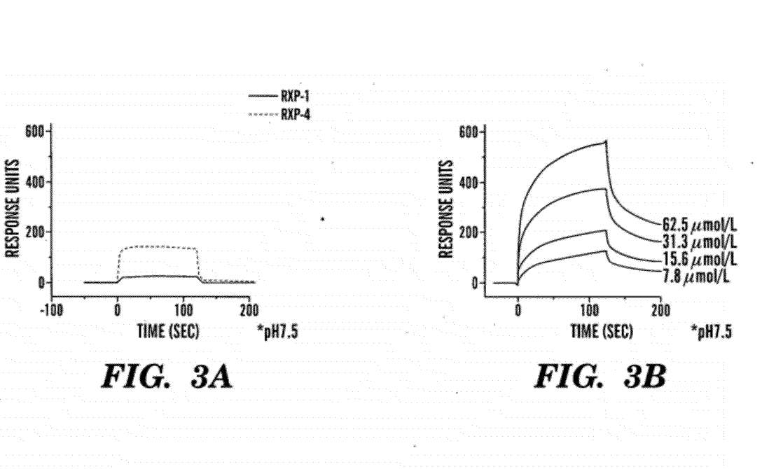

Phage display allows for the rapid screening of thousands of peptides, but the characteristics of binding cannot be properly defined. Furthermore, the peptides are part of a capsid protein, which may affect the ability of the peptide to properly interact with the target protein. Therefore, some of the peptides identified by the phage screening were selected to, using SPR, further characterize their ability to bind Cx43CT. Recombinant Cx43CT was covalently bound to a carboxymethyl dextran matrix and synthetic peptides presented for binding. A total of six 12-mer peptides from the non-biased screening (peptides labeled RXP1 to RXP6 in Table 1) and 5 peptides from the biased screening (labeled RXP-A to RXP-E in Table 3) were tested. Peptides RXP2 (SEQ ID NO:13), RXP3 (SEQ ID NO:9), RXP5 (SEQ ID NO:16), and RXP6 (SEQ ID NO:17) showed no significant binding (less than 100 RU of maximal response at peptide concentration of 1 mM). Peptide RXP1 (SEQ ID NO:12) showed a detectable, pH-dependent binding. Peptide RXP4 (SEQ ID NO:15) caused the largest deflection in SPR signal. An example is shown in FIG. 3A. RXP4 (250 μM) was added at time “zero.” Washout was initiated after two minutes of exposure. The peptide dissociated rapidly and completely, suggesting poor binding affinity to Cx43CT. Higher concentrations of the peptide were tested but an asymptotic maximum response could not be detected. In addition, dissociation rates were too fast to reliably use them to calculate the kinetics of binding. As such, these results do show Cx43CT-RXP4 binding, but the interaction between the two molecules was too weak to allow for proper calculation of kinetic values.

A different result emerged from the testing of the doublets. Peptides RXP-A (SEQ ID NO:6)-RXP-D (SEQ ID NO:9) showed no significant binding to Cx43CT. In contrast, peptide RXP-E (SEQ ID NO:10) generated a large, concentration-dependent deflection followed by a slow dissociation upon washout, as shown in FIG. 3B. The transitions were well-fit by exponential functions and the rates of association and dissociation were used as described in Example 4 to estimate the kinetic parameters. A full range of concentrations was tested in three separate occasions and at two different pHs of the solvent (6.5 and 7.4). No differences were observed as a function of pH. From these studies, a dissociation constant (K_{D}) of 3.9 μM was calculated for the interaction between Cx43CT and RXP-E. This value is similar to that measured for the association of Cx43CT to well-known binding partners such as the SH3 domain of c-src (Duffy et al., “Regulation of Connexin43 Protein Complexes by Intracellular Acidification,” Circ. Res. 94(2):215-222 (2004); Sorgen et al., “Structural Changes in the Carboxyl Terminus of the Gap Junction Protein Connexin43 Indicates Signaling between Binding Domains for c-Src and Zonula Occludens-1,” J. Biol. Chem. 279(2):54695-54701 (2004), which are hereby incorporated by reference in their entirety), and the second PDZ domain of ZO-1 (Giepmans et al., “Connexin-43 Interactions with ZO-1 and Alpha- and Beta-tubulin,” Cell Commun. Adhes. 8(4-6):219-223 (2001), which is hereby incorporated by reference in its entirety), and that measured for the binding of Aquaporin-0 to Cx45.6 (Yin et al., “The Development-associated Cleavage of Lens Connexin 45.6 by Caspase-3-like Protease is Regulated by Casein Kinase II-mediated Phosphorylation,” J. Biol. Chem. 276(37):34567-34572 (2001), which is hereby incorporated by reference in its entirety). Overall, these results show that different RXP peptides are able to interact with Cx43CT in vitro with various degrees of affinity. The possibility of structural modifications caused on Cx43CT as a result of RXP interactions (and hence the possible location of the binding site) was assessed next, as described in Example 11.

### Example 11

### Nuclear Magnetic Resonance (NMR)

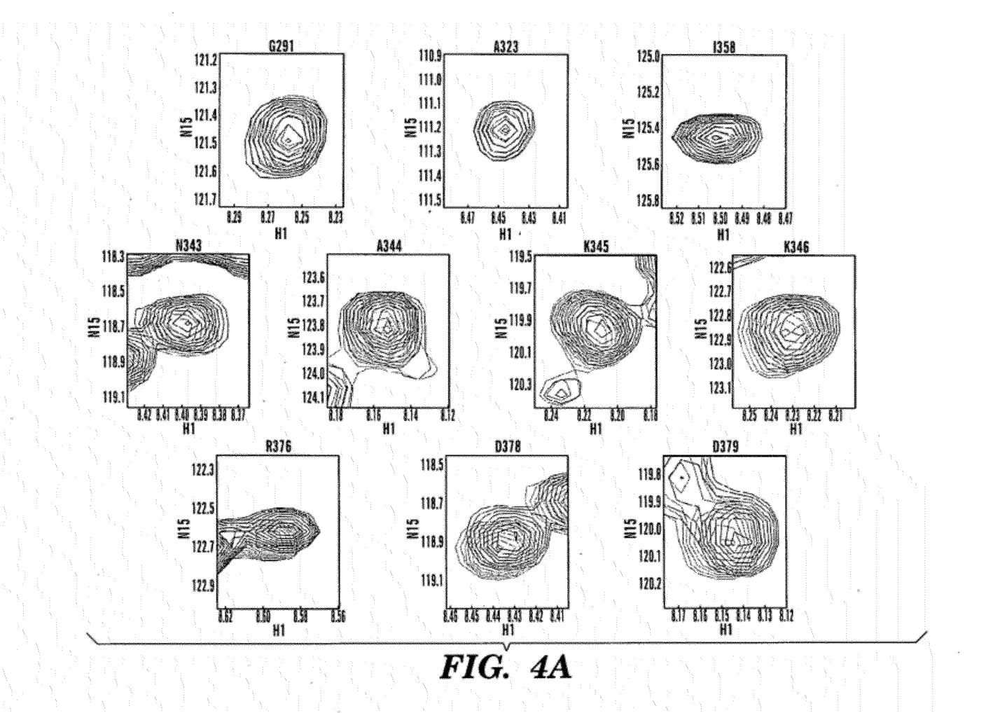

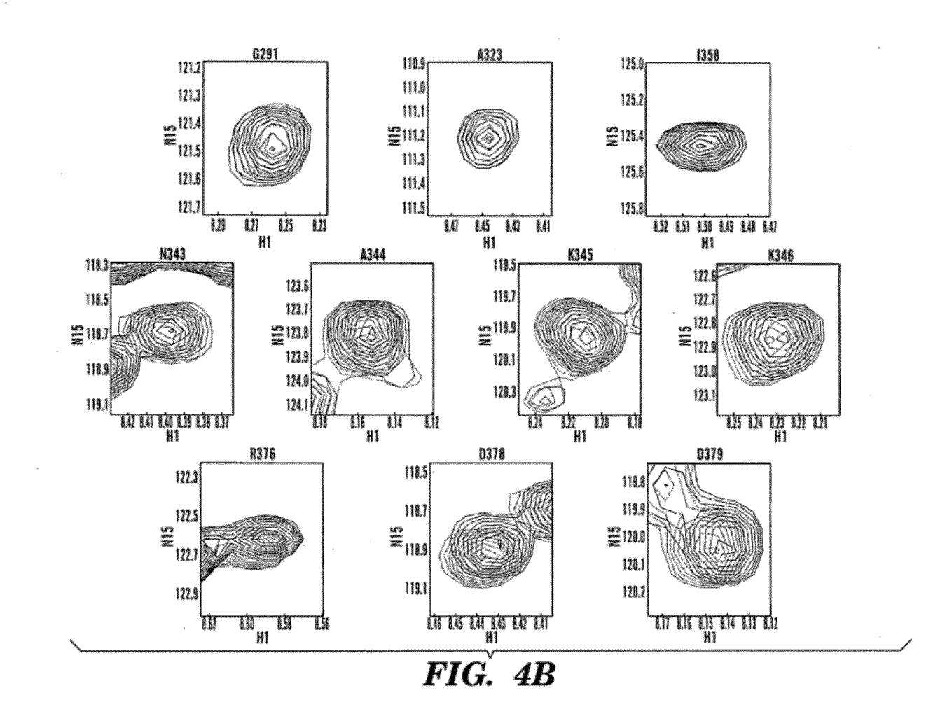

Peptides RXP1, RXP4 and RXP-E were tested for their ability to modify the structure of Cx43CT. The peptides were diluted in PBS (pH 5.8) containing ¹⁵N-Cx43CT, and ¹⁵N-HSQC spectra were acquired. Results for RXP4 and RXP-E are shown in FIGS. 4A (RXP4) and 4B (RXP-E). The specific resonance peaks that correspond to each amino acid in the Cx43CT sequence have been assigned (Sorgen et al., “Sequence-specific Resonance Assignment of the Carboxyl Terminal Domain of Connexin43,” J. Biomol. NMR 23(3):245-246 (2002), which is hereby incorporated by reference in its entirety). Accordingly, shifts in the resonance assignments directly reveal the identity of the amino acids whose position in space is modified by the presence of the peptide. As shown in FIG. 4A-B, addition of both RXP4 (FIG. 4A) and RXP-E (FIG. 4B) peptides strongly affected the resonance peaks of residues R376, D378, and D379 of Cx43CT, thus indicating the possible location for the RXP4 and RXP-E binding. In addition, there was a minor shift in amino acids 343-346. These residues are part of the α-helical domains of Cx43CT and may be involved in intramolecular interactions relevant for Cx43 regulation (Delmar et al., “Structural Bases for the Chemical Regulation of Connexin43 Channels,” Cardiovasc. Res. 62(2):268-275 (2004), which is hereby incorporated by reference in its entirety; see also Example 15). Resonance peaks for G291, A323 and I358 are presented as examples of residues whose position was unaffected by the peptides. Residues P375 and P377 do not provide an identifiable resonance peak because they do not contain an amide bond; yet, these residues may also be a part of the binding site. In contrast, no resonance shifts were observed when Cx43CT was exposed to RXP-1. This is consistent with SPR experiments showing very weak (almost undetectable) interaction between RXP-1 and Cx43CT (FIG. 3A). Overall, these results show that RXP-4 and RXP-E alter the conformation of Cx43 at residues 343 to 346 and 376 to 379.

### Example 12

### Effect of RXP-E on Cx43 Channels

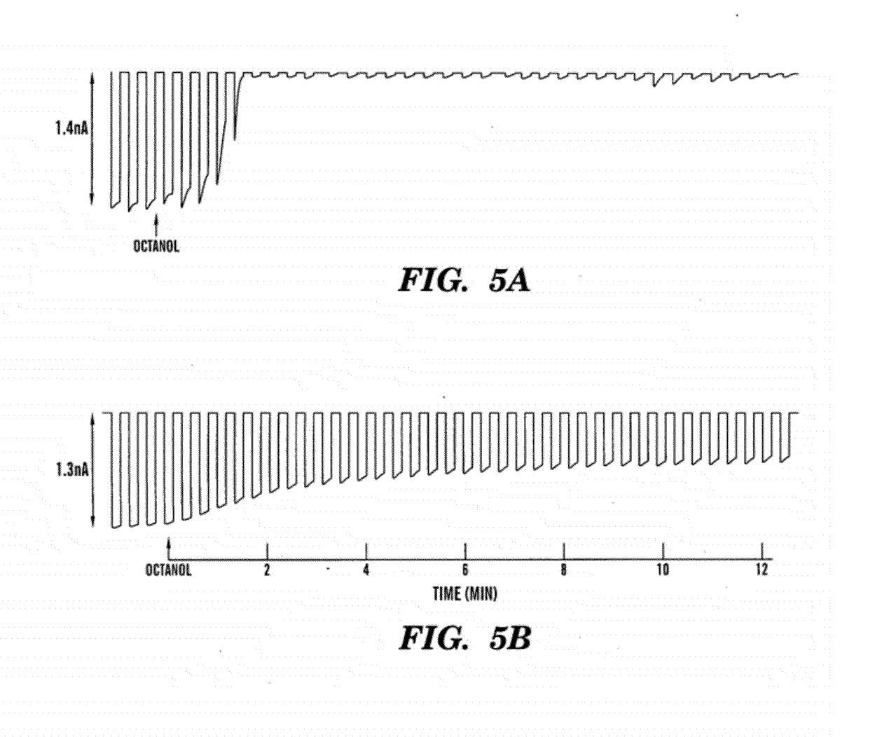

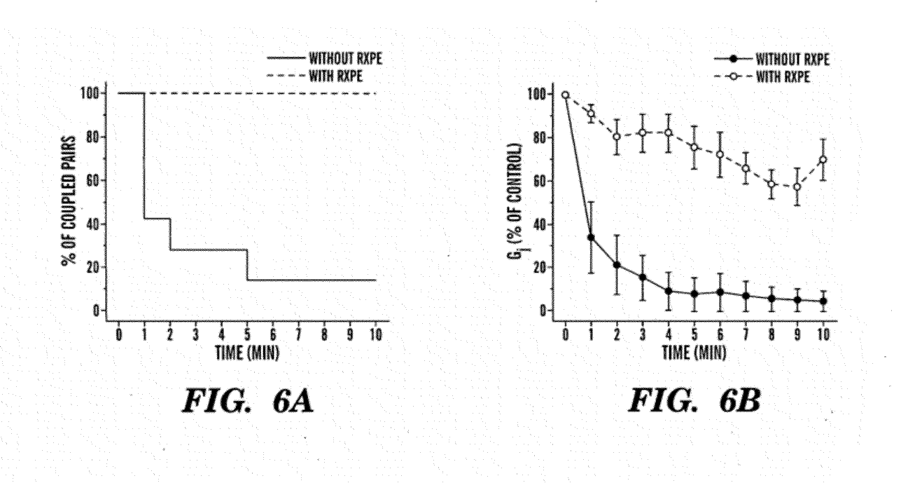

The ability of RXP-E to bind to Cx43CT suggests that this peptide may also alter the behavior of Cx43 channels. Gap junction currents were recorded from N2a cells transfected with Cx43. To reduce macroscopic currents and allow for detection of single channel events, cell pairs were superfused with octanol (Anumonwo et al., “The Carboxyl Terminal Domain Regulates the Unitary Conductance and Voltage Dependence of Connexin40 Gap Junction Channels,” Circ. Res. 88(7):666-673 (2001); Seki et al., “Modifications in the Biophysical Properties of Connexin43 Channels by a Peptide of the Cytoplasmic Loop Region,” Circ. Res. 95(4):e22-e28 (2004); Seki et al., “Loss of Electrical Communication, but Not Plaque Formation, After Mutations in the Cytoplasmic Loop of Connexin43,” Heart Rhythm. 1(2):227-233 (2004), which are hereby incorporated by reference in their entirety). The presence of RXP-E in the internal pipette solution prevented octanol-induced closure of Cx43 channels, as shown in FIG. 5A-B. FIG. 5A depicts junctional current traces obtained from a Cx43-expressing cell pair under control conditions (no RXP-E). Transjunctional voltage (Vⱼ) was +60 mV. Octanol was added to the superfusate at the point indicated by the arrow. After a short delay, junctional current abruptly decreased, reaching zero within three minutes after onset of octanol. The ability of octanol to uncouple gap junctions with a high degree of efficiency has been extensively reported in the literature (Johnston & Ramon, “Electrotonic Coupling in Internally Perfused Crayfish Segmented Axons,” J. Physiol. 317:509-518 (1981), which is hereby incorporated by reference in its entirety). The traces shown in FIG. 5B were obtained from a different cell pair. Here, RXP-E was diluted in the internal pipette solution. Three minutes after onset of octanol, only a minor decrease in Gⱼ was observed. Cumulative data are presented in FIG. 6A-B. FIG. 6A depicts the percent of cell pairs that remained coupled following the onset of octanol superfusion (uncoupling defined as zero junctional current elicited by a 60-mV transjunctional voltage pulse). Ten different cell pairs were recorded using patch pipettes filled with a solution containing RXP-E. Seven pairs were tested without RXP-E. Control experiments (no RXP-E) were conducted on cells in the same plate as those that failed to uncouple in the presence of the peptide. As shown in FIG. 6A, none of the cell pairs exposed to RXP-E uncoupled following octanol (discontinuous line), whereas 6 out of 7 control cell pairs in absence of RXP-E (continuous line) showed complete uncoupling after 5 minutes of octanol superfusion. FIG. 6B shows the time course of changes in junctional conductance (Gⱼ; measured relative to control for each individual experiment) as a function of time after onset of octanol. Data obtained in the absence of RXP-E is depicted by a continuous line and closed symbols. The results show the characteristic rapid drop in Gⱼ, reaching an asymptotic value after approximately 5 minutes of octanol superfusion. The broken line and open symbols correspond to data obtained in the presence of RXP-E. Though no uncoupling was observed, a slight decrease in Gⱼ was apparent. On average, Gⱼ reached a minimum of 57.6±8.6% from control within 9 minutes after the beginning of octanol superfusion. Overall, the data show that RXP-E prevented octanol-induced uncoupling in Cx43-expressing cell pairs. The RXP-E effect is not ascribable to the presence of any peptidic molecule in the patch pipette. Indeed, previous studies have shown that a peptide derived from the cytoplasmic loop altered channel characteristics but did not alter octanol-sensitivity (Seki et al., “Modifications in the Biophysical Properties of Connexin43 Channels by a Peptide of the Cytoplasmic Loop Region,” Circ. Res. 95(4):e22-e28 (2004), which is hereby incorporated by reference in its entirety).

### Example 13

### Effect of RXP-E on Truncated Cx43 Channels

The results presented in FIGS. 2A-4B show that RXP-E binds to Cx43CT, suggesting that the effect of RXP-E requires the integrity of the CT domain.

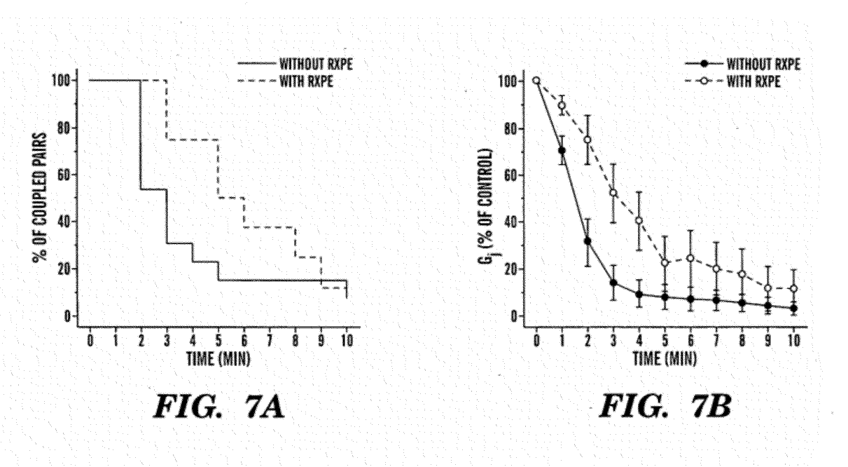

To assess this hypothesis, the effect of RXP-E on octanol-induced uncoupling between cells expressing a truncated form of Cx43 lacking most of the CT domain (mutant M257; see Morley et al., “Intramolecular Interactions Mediate pH Regulation of Connexin43 Channels,” Biophys. J. 70(3):1294-1302 (1996), which is hereby incorporated by reference in its entirety) was tested using the procedure described in Example 12. Results are shown in FIG. 7A-B. In this case, octanol led to complete uncoupling regardless of the presence of the peptide, as shown in FIG. 7A, though there was a slight delay in the time course of the reduction in Gⱼ, as shown in FIG. 7B. The results suggest that a minor effect of RXP-E may be preserved after truncation of the CT domain, though the full effect is largely dependent on the integrity of the CT domain.

### Example 14

### RXP-E Partially Prevents Acidification-Induced Uncoupling

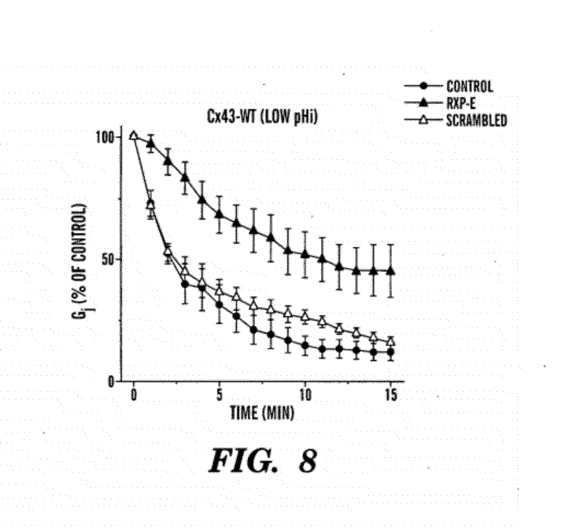

Whether RXP-E can interfere with Cx43 regulation by factors with some possible pathophysiological relevance was next assessed. To address this question, the extent and time course of uncoupling induced by reduced intracellular pH (“pHᵢ”) was characterized. Patch pipettes were filled with an MES-containing solution, buffered to a pH of 6.2. Junctional current (Iⱼ) was measured immediately after patch break and every 20 seconds thereafter. FIG. 8 shows these results. In the absence of RXP-E, Gⱼ decreased progressively, reaching 15.0±2.9% of control within 15 minutes after patch break (continuous line and closed symbols; N5). The line with the closed triangles represents the average data obtained when RXP-E was present in the pipette solution (N=6). Clearly, the initial drop in Gⱼ was similar to that observed in control. However, the progression of uncoupling was interrupted and Gⱼ decreased only to 57.0±5.8% of control. The presence of a control peptide (open triangles) did not alter pHi mediated uncoupling. The data show that RXP-E partially prevented the closure of Cx43 channels consequent to a reduction in pHᵢ.

### Example 15

### Effect of RXP-E on Single Channel Activity

Cx43 channels can reside in at least three distinct states: closed, open and residual (see Harris, “Emerging Issues of Connexin Channels: Biophysics Fills the Gap,” Q. Rev. Biophys. 34(3):325-472 (2001), 35(1):109 (2002) (erratum), which is hereby incorporated by reference in its entirety). Open-to-residual transitions are considered responsible for “fast Vⱼ gating” (i.e., the rapid, voltage-dependent component of junctional current inactivation seen in Cx43 and other connexin channels) (Moreno et al., “Role of the Carboxyl Terminal of Connexin43 in Transjunctional Fast Voltage Gating,” Circ. Res. 90(4):450-457 (2002), 92(1):e30 (2003) (erratum); Harris, “Emerging Issues of Connexin Channels: Biophysics Fills the Gap,” Q. Rev. Biophys. 34(3):325-472 (2001), 35(1):109 (2002) (erratum), which are hereby incorporated by reference in their entirety).

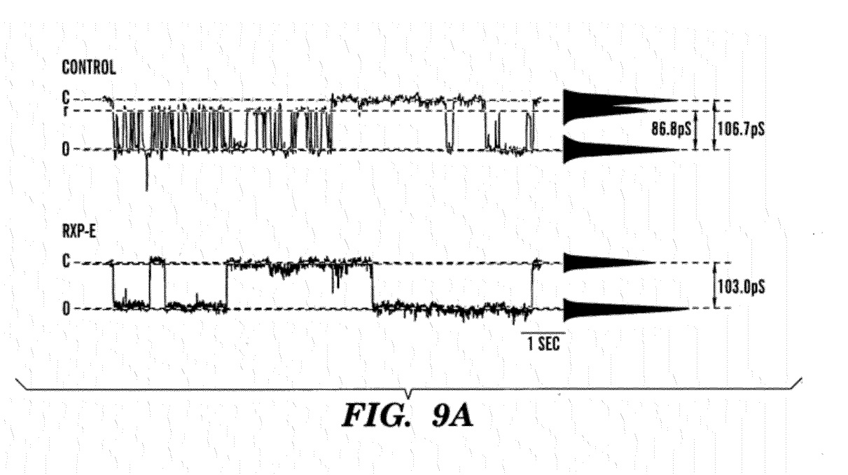

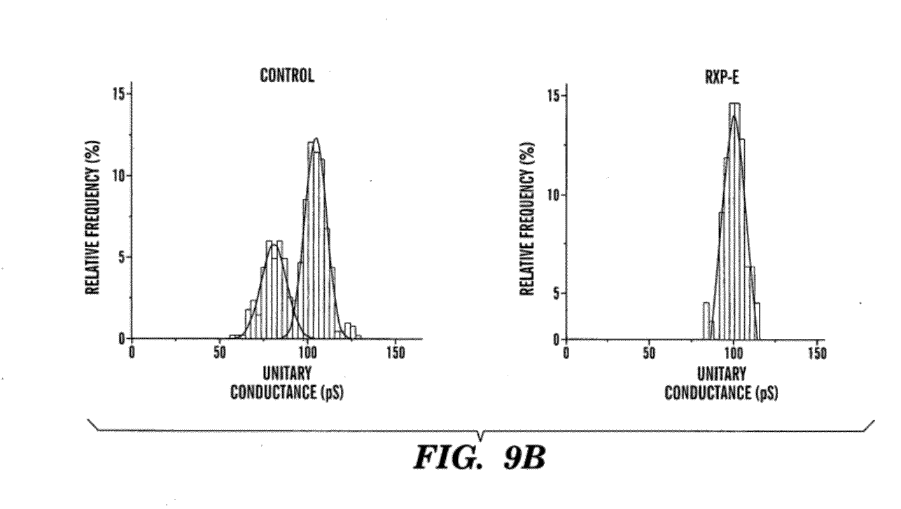

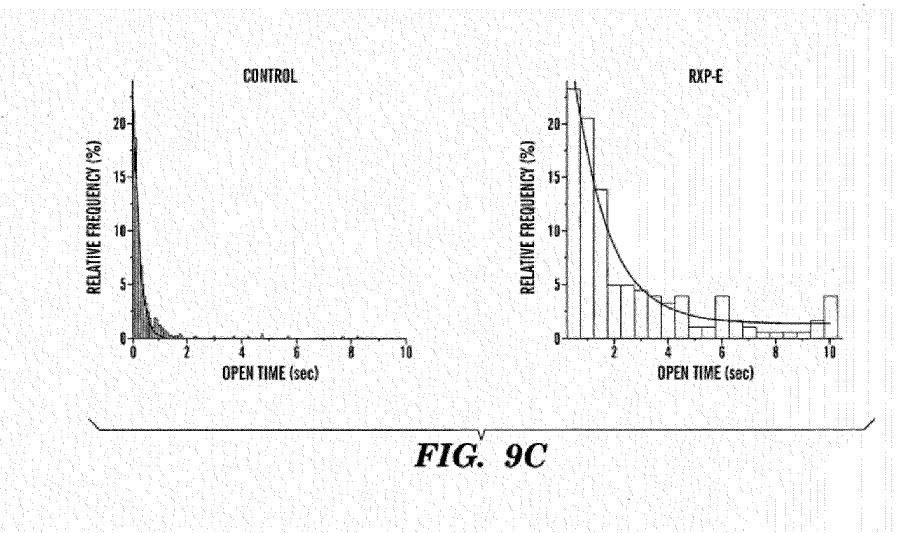

Spontaneous single channel activity from four Cx43-expressing cell pairs was recorded. The single channel traces in the presence of RXP-E are shown in FIGS. 9A-C. Transjunctional voltage (Vⱼ) was +60 mV. The horizontal dotted lines indicate the current levels corresponding to the closed (C) and open (O) configurations. For the trace shown in FIG. 9A (top), pipettes were filled with internal pipette solution buffered at pH 7.2. After an initial, spontaneous decrease in Gⱼ from 0.56 to 0.1 nS, single channel events were resolvable. The second trace in FIG. 9A was recorded from another pair, using patch pipettes containing an internal solution buffered to pH 6.2. Single channel events were seen immediately after patch break. Overall, the events recorded showed a unitary conductance similar to that in the absence of RXP-E, though open times were greatly prolonged and the residual state was noticeably absent. Cumulative data are shown in FIG. 9B-C. FIG. 9B shows a histogram of the measured values of unitary conductance. The Gaussian distribution centered at 100.5 pS, not different from what has been reported for wild-type Cx43 channels (Moreno et al., “Role of the Carboxyl Terminal of Connexin43 in Transjunctional Fast Voltage Gating,” Circ. Res. 90(4):450-457 (2002), 92(1):e30 (2003) (erratum); Elenes et al., “Heterotypic Docking of Cx43 and Cx45 Connexons Blocks Fast Voltage Gating of Cx43,” Biophys. J. 81(3):1406-1418 (2001); Bukauskas et al., “Conductance and Permeability of the Residual State of Connexin43 Gap Junction Channels,” J. Gen. Physiol. 119(2):171-185 (2002), which are hereby incorporated by reference in their entirety). A single peak was detected, consistent with the observation that no residual state was observed. Histograms of open times in the absence and presence of RXP-E are presented in FIG. 9C. An average of the open times measured in the presence of RXP-E yielded a value of 1.27 seconds (N=3, n=368). The latter contrasts with measurements obtained from Cx43 channels in the absence of RXP-E of 0.12 seconds (N=3, n=475), but is consistent with the reported mean open time of Cx43 channels (126±20 ins (Moreno et al., “Role of the Carboxyl Terminal of Connexin43 in Transjunctional Fast Voltage Gating,” Circ. Res. 90(4):450-457 (2002), 92(1):e30 (2003) (erratum), which is hereby incorporated by reference in its entirety)). Overall, the data suggest that RXP-E greatly increases the stability of the open state of the channel, without modifying its unitary conductance.

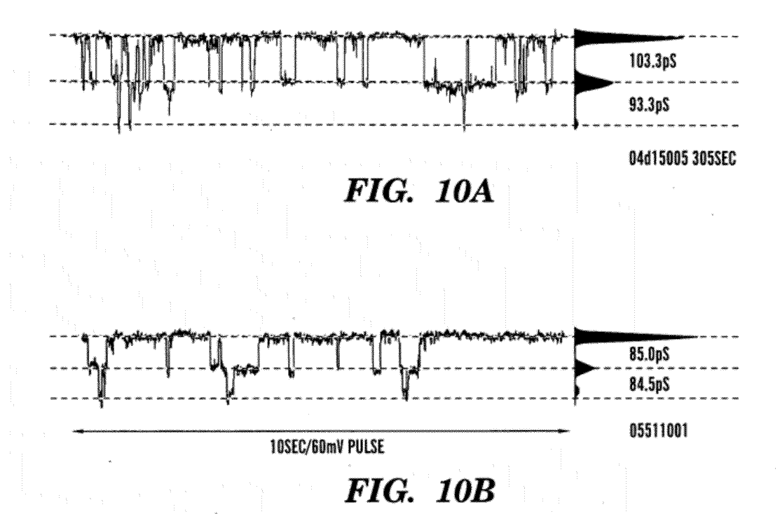

Additional experiments were conducted on cell pairs expressing M257 channels. The results obtained in the absence of RXP-E are shown in FIG. 10A. Previous studies have shown that truncation of the CT domain leads to a prolongation of the mean open time (Moreno et al., “Role of the Carboxyl Terminal of Connexin43 in Transjunctional Fast Voltage Gating,” Circ. Res. 90(4):450-457 (2002), 92(1):e30 (2003) (erratum)). A similar result was observed in these experiments. However, in contrast with the results observed for the full-length channel, rapid and frequent transitions between states were consistently recorded in the presence of RXP-E, as shown in FIG. 10B. These results indicate that the ability of RXP-E to increase open time in Cx43 channels requires the integrity of the CT domain.

A high throughput assay to identify peptidic sequences that bind to Cx43CT has been carried out. These studies led to the characterization of a group of peptides sharing an “RXP” motif. A common feature on these peptides was an excess of basic residues within the sequence. The binding kinetics and structural modification of CT were studied in two of these peptides; one peptide was tested for its effect on Cx43 channel function.

Technical considerations. Phage display is a powerful screening technique, and like any high-throughput method, it is prone to both false positive and false negative results. It is possible that due to experimental conditions, sequences that would be of relevance were not identified. Similarly, it is possible that some of the sequences identified do not represent good binders when isolated from the phage and presented to the Cx43CT in a different environment. Despite this limitation, the system provided the ability to recognize a group of molecules with the potential to significantly modify channel behavior, as described in Examples 12-15 and shown in FIGS. 5A-9C. Thus, while potential Cx43 binding sequences were likely missed, some of those identified provided an interesting system for the study of Cx43CT regulation and Cx43 function. It is also recognized that, as any in vitro system, SPR and NMR carry the risk of not accurately representing the interaction between molecules in the microenvironment of the cell. However, there is good correlation between in vitro interactions involving Cx43CT and those demonstrated from cellular preparations or functional assays (Duffy et al., “pH-Dependent Intramolecular Binding and Structure Involving Cx43 Cytoplasmic Domains,” J. Biol. Chem. 277(39):36706-36714 (2002); Duffy et al., “Regulation of Connexin43 Protein Complexes by Intracellular Acidification,” Circ. Res. 94(2):215-222 (2004); Giepmans et al., “Interaction of c-Src with gap junction protein connexin-43. Role in the Regulation of Cell-cell Communication,” J. Biol. Chem. 276(11):8544-8549 (2001); Seki et al., “Modifications in the Biophysical Properties of Connexin43 Channels by a Peptide of the Cytoplasmic Loop Region,” Circ. Res. 95(4):e22-e28 (2004); Sorgen et al., “Structural Changes in the Carboxyl Terminus of the Gap Junction Protein Connexin43 Indicates Signaling between Binding Domains for c-Src and Zonula Occludens-1,” J. Biol. Chem. 279(2):54695-54701 (2004), which are hereby incorporated by reference in their entirety). The present invention shows that the molecule with the highest binding affinity—as tested by SPR—also had a significant effect on Cx43CT structure and on channel function. Thus, within the limitations implicit to every in vitro study, a combination of in vitro methods led to the identification of molecules with potential biological relevance.

Dual patch clamp was used in a whole-cell configuration to deliver RXP-E to the intracellular space and assess the consequence of RXP-E on Cx43 function. The effect of a peptide of the cytoplasmic loop on channel function has previously been characterized using this method (Seki et al., “Modifications in the Biophysical Properties of Connexin43 Channels by a Peptide of the Cytoplasmic Loop Region,” Circ. Res. 95(4):e22-e28 (2004), which is hereby incorporated by reference in its entirety). While this is certainly an effective method of peptide delivery, the possible experiments are limited, and constrained to cell pairs. Current studies are aimed at developing methods to efficiently transfer these peptides into intact cells.

Selection of peptides. The phage display results suggested that Cx43CT had a preference for peptides with an excess of basic residues, which may be associated with their positive charge. Though the pKa of histidine is near the pHs used for binding in the present study, the probability of the imidazole ring of histidine being protonated at the pHs tested is certainly higher than that of any other amino acid, with the exception of lysine and arginine. A preference on the position of the basic residues within the primary sequence of the peptide was not observed. However, it remains to be tested whether there is a conservation of secondary structure not apparent from the primary sequence. Further NMR studies will be directed at determining the structure of RXP peptides and identifying relevant structures that may facilitate the binding to Cx43CT.

Screening of the biased pre-bound library failed to identify any double sequences. Yet, the vast majority of bound RXP peptides corresponded to doublets, which suggests that Cx43CT had a strong preference for longer sequences. Whether this is consequent to the increased number of “RXP” motifs, the increased number of basic residues (note that at least some of the linkers were also rich in basic amino acids), the increased size of the peptide, or any combination of the above, remains to be determined. Clearly, among the “double RXP” series, the one peptide with the highest binding affinity and more significant effect on channel function was found.

The mechanism underlying doublet formation can be deduced from the analysis of the sequences of the linkers. To produce phage with random inserts, a single stranded oligonucleotide containing the randomized sequence must be replicated using the Klenow fragment of DNA polymerase. This forms a double stranded DNA fragment with blunt ends. Restriction digestion puts “sticky ends” on this fragment for its insertion into the phage DNA. In rare cases, one of these “sticky ends” was either not produced due to incomplete digestion, or filled after digestion due to continued activity of the DNA polymerase. This led to blunt ends capable of ligation, rather than mismatched “sticky ends.” These events were rare enough as to not be detected in the sampling of the library sequences performed prior to screening.

The peptides most frequently repeated in the phage display screening were not the ones with the highest binding affinity when tested by SPR. This apparent discrepancy may result from the fact that codon distribution (and, consequently, tRNA availability) is not equal in the bacteria used to amplify the phage. Accordingly, even if a peptide binds with high affinity, its amplification may be limited by the presence of rare codons in the phage sequence. Consistent with this hypothesis, RXP-E contained the least represented codon in E. coli, likely acting as a limiting factor in its production. The latter emphasizes not only this specific limitation of the phage display method but also the importance of using alternative in vitro techniques to assess binding by the peptides identified through the screening process.

Structural modifications in Cx43CT. Structural analysis of Cx43CT revealed that both RXP4 and RXP-E caused a shift in the resonance peaks of amino acids 376, 378, and 379. Positions 375 and 377 are occupied by proline residues, which are not detected by the ¹⁵N-HQS experiment (Sorgen et al., “Sequence-specific Resonance Assignment of the Carboxyl Terminal Domain of Connexin43,” J. Biomol. NMR 23(3):245-246 (2002), which is hereby incorporated by reference in its entirety). It is thus possible that the region structurally modified by RXP peptides extends between 375 and 379. The resonance shifts strongly suggest that those amino acids are directly involved in peptide binding. Interestingly, this region of Cx43CT is within the PDZ binding domain (Duffy et al., “pH-Dependent Intramolecular Binding and Structure Involving Cx43 Cytoplasmic Domains,” J. Biol. Chem. 277(39):36706-36714 (2002); Giepmans & Moolenaar, “The Gap Junction Protein Connexin43 Interacts with the Second PDZ Domain of the Zona Occludens-1 Protein,” Curr. Biol. 8(16):931-934 (1998); Sorgen et al., “Structural Changes in the Carboxyl Terminus of the Gap Junction Protein Connexin43 Indicates Signaling between Binding Domains for c-Src and Zonula Occludens-1,” J. Biol. Chem. 279(2):54695-54701 (2004), which are hereby incorporated by reference in their entirety) and near areas relevant for Cx43 phosphorylation (Duffy et al., “pH-Dependent Intramolecular Binding and Structure Involving Cx43 Cytoplasmic Domains,” J. Biol. Chem.

277(39):36706-36714 (2002); Lampe et al., “Phosphorylation of Connexin43 on Serine-368 by Protein Kinase C Regulates Gap Junctional Communication,” J. Cell Biol. 149(7):1503-1512 (2000); Sorgen et al., “Structural Changes in the Carboxyl Terminus of the Gap Junction Protein Connexin43 Indicates Signaling between Binding Domains for c-Src and Zonula Occludens-1,” J. Biol. Chem. 279(2):54695-54701 (2004), which are hereby incorporated by reference in their entirety). Moreover, both peptides also modified the position of residues within the second alpha helical domain of Cx43CT (Delmar et al., “Structural Bases for the Chemical Regulation of Connexin43 Channels,” Cardiovasc. Res. 62(2):268-275 (2004), which is hereby incorporated by reference in its entirety), which is involved in pH-dependent dimerization of the protein (Sorgen et al., “pH-Dependent Dimerization of the Carboxyl Terminal Domain of Cx43,” Biophys. 187(1):574-581 (2004), which is hereby incorporated by reference in its entirety). As such, RXP peptides may interfere with both intra- and inter-molecular interactions that could regulate the function of a gap junction channel.

Octanol-induced uncoupling and the effect of RXP-E. RXP-E prevented octanol-induced uncoupling, and the full effect required the integrity of the CT domain. The molecular mechanism by which octanol causes gap junction closure is not completely understood. It has been proposed that volatile anesthetics and long-chain alcohols exert their effects on membrane channels (including gap junctions) via a non-specific action on the lipid bilayers (Takens-Kwak et al., “Mechanism of Heptanol-induced Uncoupling of Cardiac Gap Junctions: A Perforated Patch-clamp Study,” Am. J. Physiol. 262(6):C1531-C1538 (1992); Bastiaanse et al., “Heptanol-induced Decrease in Cardiac Gap Junctional Conductance is Mediated by a Decrease in the Fluidity of Membranous Cholesterol-rich Domains,” J. Membr. Biol. 136(2):135-145 (1993), which are hereby incorporated by reference in their entirety). However, recent studies on ligand-gated channels have challenged the latter hypothesis and shown that n-alkanols exert their functional effect by interacting with specific binding pockets in the channel proteins (Mascia et al., “Specific Binding Sites for Alcohols and Anesthetics on Ligand-gated Ion Channels,” Proc. Nat'l Acad. Sci. USA 97(16):9305-9310 (2000), which is hereby incorporated by reference in its entirety). Additional studies indicate that octanol causes Cx50 hemichannel closure, but only when the alcohol is presented at the extracellular side of the channel (Eskandari et al., “Inhibition of Gap Junction Hemichannels by Chloride Channel Blockers,” J. Membr. Biol. 185(2):93-102 (2002), which is hereby incorporated by reference in its entirety). The latter would argue against the concept of an effect purely dependent on membrane fluidity; instead, the results support the notion that octanol may interact with a specific region of the channel protein and that such an interaction may be responsible (at least in part) for the closure of the channel.

Previous studies indicate that octanol causes a reduction in gap junction channel open probability, without affecting unitary conductance (Seki et al., “Modifications in the Biophysical Properties of Connexin43 Channels by a Peptide of the Cytoplasmic Loop Region,” Circ. Res. 95(4):e22-e28 (2004), which is hereby incorporated by reference in its entirety). Interestingly, RXP-E seems to have a significant, CT-dependent effect at stabilizing the channel in its open conformation (see FIGS. 9A and 9C). These two phenomena may be related, and RXP-E may act by counteracting the ability of octanol to bring the channel into the closed state.

Previous studies show that the CT domain is not a part of the pore-forming region of the channel. Furthermore, in the absence of CT, octanol still induces channel closure. Hence, the question remains as to how by binding to the CT domain, RXP-E can prevent octanol-induced closure. Cx43CT interacts with regions of the channel affiliated with the pore (Duffy et al., “pH-Dependent Intramolecular Binding and Structure Involving Cx43 Cytoplasmic Domains,” J. Biol. Chem. 277(39):36706-36714 (2002); Seki et al., “Modifications in the Biophysical Properties of Connexin43 Channels by a Peptide of the Cytoplasmic Loop Region,” Circ. Res. 95(4):e22-e28 (2004), which are hereby incorporated by reference in their entirety). It is therefore possible that RXP-E may use the CT as a “scaffolding,” from which it can also interact with pore-forming or pore-vestibular regions (including the CL domain; see Duffy et al., “pH-Dependent Intramolecular Binding and Structure Involving Cx43 Cytoplasmic Domains,” J. Biol. Chem. 277(39):36706-36714 (2002); Seki et al., “Modifications in the Biophysical Properties of Connexin43 Channels by a Peptide of the Cytoplasmic Loop Region,” Circ. Res. 95(4):e22-e28 (2004); Delmar et al., “Structural Bases for the Chemical Regulation of Connexin43 Channels,” Cardiovasc. Res. 62(2):268-275 (2004), which are hereby incorporated by reference in their entirety) thereby holding the channel in its open state.

Effect of RXP-E on pH gating of Cx43. Though the effect of RXP-E on octanol-induced uncoupling is interesting, its functional implications may be limited. Of more relevance to pathophysiology is the observation that RXP-E partially prevented acidification-induced uncoupling. Future experiments are needed to demonstrate that RXP-E interferes with pH gating of native gap junctions in Cx43-expressing tissues (such as heart or astrocytes). In addition, the possible effects of RXP-E on other membrane channels need to be determined. The latter notwithstanding, in the particular case of the heart, acidification-induced uncoupling is considered one of the possible substrates for ventricular arrhythmias following myocardial ischemia or infarction (Cascio, “Myocardial Ischemia: What Factors Determine Arrhythmogenesis?,” J. Cardiovasc. Electrophysiol. 12(6):726-729 (2001), which is hereby incorporated by reference in its entirety). Yet, the extent to which pH gating of Cx43 is beneficial or deleterious to the function of the ischemic heart remains undetermined. By interfering with gap junction regulation, RXP-E (or future derivatives of it) may serve as tools to dissect the specific role that gap junction regulation plays in determining the electrophysiological profile of the ischemic heart.

Future implications. Peptides or peptide-derived molecules have been used in the past in an attempt to regulate Cx43. A number of those sequences have been derived from Cx43 itself (Calero et al., “A 17mer Peptide Interferes with Acidification-induced Uncoupling of Connexin43,” Circ. Res. 82(9):929-935 (1998); Seki et al., “Modifications in the Biophysical Properties of Connexin43 Channels by a Peptide of the Cytoplasmic Loop Region,” Circ. Res. 95(4):e22-e28 (2004), which are hereby incorporated by reference in their entirety). Others, though apparently capable of preserving intercellular coupling under certain conditions (Xing et al., “ZP123 Increases Gap Junctional Conductance and Prevents Reentrant Ventricular Tachycardia During Myocardial Ischemia in Open Chest Dogs,” J. Cardiovasc. Electrophysiol. 14(5):510-520 (2003); Eloff et al., “Pharmacological Modulation of Cardiac Gap Junctions to Enhance Cardiac Conduction: Evidence Supporting a Novel Target for Antiarrhythmic Therapy,” Circulation 108(25):3157-3163 (2003), which are hereby incorporated by reference in their entirety), seem to act indirectly via modulation of molecules (such as kinases) that in turn regulate Cx43 (Weng et al., “Pharmacological Modification of Gap Junction Coupling by an Antiarrhythmic Peptide via Protein Kinase C Activation,” FASEB J. 16(9):1114-1116 (2002); Dhein et al., “Protein Kinase Cα Mediates the Effect of Antiarrhythmic Peptide on Gap Junction Conductance,” Cell Commun. Adhes. 8(4-6):257-264 (2001), which are hereby incorporated by reference in their entirety). The latter carries a high risk of connexin-unrelated effects on cell function, as kinases are likely to interact with a variety of molecules, not only Cx43. The present invention presents the first demonstration of a peptidic molecule, exogenous to the cell, which can prevent a form of chemically-induced uncoupling likely by direct interaction with Cx43CT. These studies enable the development of future molecules with various degrees of affinity and specificity that can alter Cx43 regulation by direct interaction with the pertinent protein domains.

In summary, a high throughput screening method combined with spectroscopic techniques has been used in the present invention to identify peptides that bind to the regulatory domain of Cx43. These results yielded a number of novel sequences, one of which prevented octanol-induced uncoupling. This is the first description of a molecule that, though carrying a sequence not present in endogenous proteins, can bind Cx43 and affect function. This is also the first description of a molecule that can prevent the closure of the channels through a direct interaction with the regulatory domain. Much remains to be learned about the effects of these peptides on Cx43, other connexins or other channels. The present description opens the door for future experiments that can lead to the development of chemical tools to regulate the function of Cx43-containing gap junctions both in health and in disease.

### Example 16

### Additional RXP Peptides

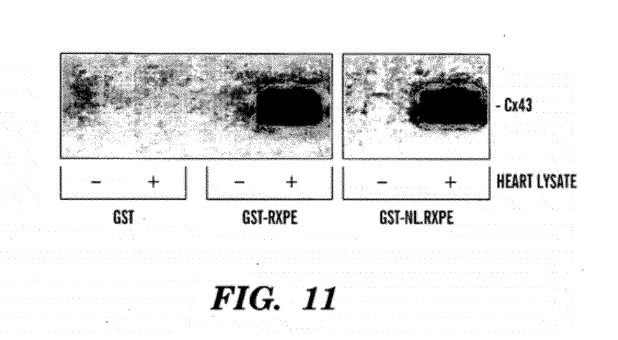

cDNA coding for SDDLRSPQLHNHMVRRKPRNPR (SEQ ID NO:11) (“NL.RXPE”) was cloned in-frame with the cDNA for glutathione S-transferase (“GST”). The resulting fusion protein, GST-NL.RXPE was produced in bacteria, and purified by conventional methods. GST-NL.RXPE was bound to glutathione beads and exposed to a rat heart lysate. After washing of the supernatant, the pellets were heated to separate the proteins from the beads. Two additional proteins were produced in parallel, and used as control: GST alone (not fused to any peptide) and GST fused to RXPE. After exposure to heart lysate, the resulting complexes that were “fished out” by the bait proteins were run on SDS-PAGE, transferred to a nitrocellulose membrane, and probed with an antibody for Cx43. As expected, as shown in FIG. 11, Cx43 was recovered when heart lysate was exposed to NL.RXPE. These results demonstrate that NL.RXPE can interact with cardiac Cx43. It is expected that other peptides of formula A-Aₙ interact with Cx43, and may be used in the methods of the present invention.

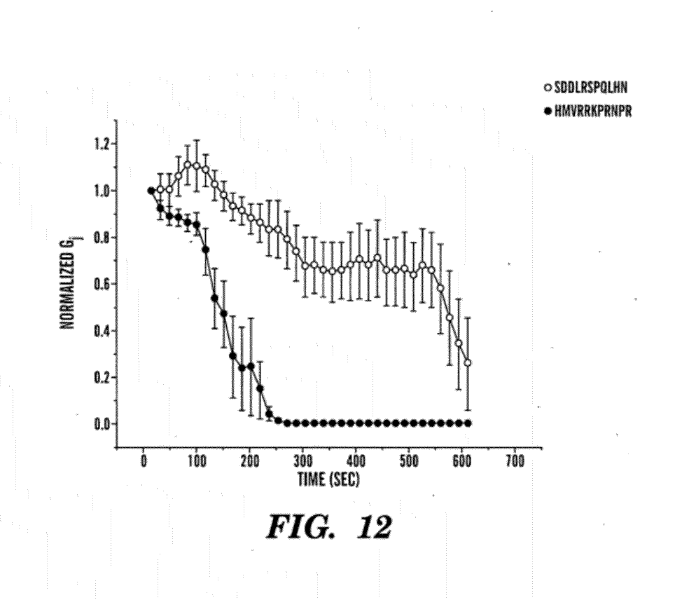

Cells were treated with heptanol to reduce coupling and with NL.RXPE. As shown in FIG. 12, the cell pairs with the peptide uncoupled at a much slower rate than those without the peptide.

### Example 17

### Small RXP Peptides

Additional peptides of decreasing length, shown in Table 5, were generated. The peptides were tested for binding to the carboxyl terminal domain of Cx43 using Surface Plasmon Resonance (“SPR”). SPR is a method that was used to identify the original RXPE sequence (Shibayama et al., “Identification of a Novel Peptide that Interferes with the Chemical Regulation of Connexin-43,” Circ. Res. 98:1365-72 (2006), which is hereby incorporated by reference in its entirety). The concentration of the peptide, relative to the amplitude of the response in arbitrary units, is shown in Table 5 (the amplitude of the response is also a function of the mass of the ligate, in this case, the molecular weight of the RXP peptide). The data show that RXP peptides as small as four amino acids in length are able to bind to the carboxyl terminal domain of Cx43; it is expected that these small RXP peptides may be used in the methods of the present invention.

TABLE 5
Small RXP Peptides

| Identification | | Molecular | | Response |
|---|---|---|---|---|
| No. | Sequence | Weight | Concentration | Dose |
| 2503 | AcRRKPRRKP- | 1134.4 | 10 mM | 294.7 |
| | NH₂ (SEQ ID | | 500 μM | 214.8 |
| | NO: 28) | | 250 μM | 147.0 |
| | | | 100 μM | 80.6 |
| | | | 25 μM | 27.5 |
| 2501 | AcRNPRNP- | 793.9 | 1.0 mM | 24.8 |
| | NH₂ (SEQ ID | | 500 μM | 14.4 |
| | NO: 29) | | | |
| 2504 | AcRRKP-NH₂ | 596.7 | 1.0 mM | 46.5 |
| | (SEQ ID | | | |
| | NO: 30) | | | |
| 2505 | AcRRNP-NH₂ | 582.7 | 1.0 mM | 34.9 |
| | (SEQ ID | | | |
| | NO: 31) | | | |
| 2502 | AcRNP-NH₂ | 426.5 | 1.0 mM | 4.6 |

### Example 18

### General Peptide Synthesis

### Apparatus and Synthetic Strategy

Peptides described in Examples 1-17 were synthesized batchwise in a polyethylene vessel equipped with a polypropylene filter for filtration using 9-fluorenylmethyloxycarbonyl (“Fmoc”) an N-α-amino protecting group and suitable common protection groups for side-chain functionalities.

### Solvents

Solvent DMF (N,N-dimethylformamide, Riedel de-Häen, Germany) was purified by passing through a column packed with a strong cation exchange resin (Lewatit S100 MB/H strong acid, Bayer AG Leverkusen, Germany) and analyzed for free amines prior to use by addition of 3,4-dihydro-3-hydroxy-4-oxo-1,2,3-benzotriazine (“Dhbt-OH”), which gives rise to a yellow color (Dhbt-O⁻ anion) if free amines are present. Solvent DCM (dichloromethane, analytical grade, Riedel de-Häen, Germany) was used directly without purification. Acetonitril (HPLC-grade, Lab-Scan, Dublin Ireland) was used directly without purification.

### Amino Acids

Fmoc-protected amino acids were purchased from Advanced ChemTech in suitable side-chain protected forms.

### Coupling Reagents

Coupling reagent diisopropylcarbodiimide (“DIC”) was purchased from (Riedel de-Häen, Germany); PyBop was purchases from Advanced ChemTech.

### Linkers

(4-Hydroxymethylphenoxy)acetic acid (“HMPA”), was purchased from Novabiochem, Switzerland, and was coupled to the resin as a preformed 1-hydroxybenzotriazole (“HOBt”) ester generated by means of DIC.

### Solid Supports

Peptides were synthesized according to the Fmoc-strategy on TentaGel S resins 0.22-0.31 mmol/g (TentaGel-S—NH₂; TentaGel S-Ram, Rapp polymere, Germany).

### Catalysts and Other Reagents

Diisopropylethylamine (“DIEA”) was purchased from Aldrich, Germany, ethylenediamine from Fluka, and piperidine and pyridine from Riedel-de Häen, Frankfurt, Germany. 4-(N,N-Dimethylamino)pyridine (“DMAP”) was purchased from Fluka, Switzerland, and used as a catalyst in coupling reactions involving symmetrical anhydrides. Ethandithiol was purchased from Riedel-de Häen, Frankfurt, Germany. 3,4-Dihydro-3-hydroxy-4-oxo-1,2,3-benzotriazine (“Dhbt-OH”), 1-hydroxybenzotriazole (“HOBt”), and 1-hydroxy-7-azabenzotriazole (“HOAt”) were obtained from Fluka, Switzerland.

### Coupling Procedures

The first amino acid can be coupled as a symmetrical anhydride in DMF generated from the appropriate n-α-protected amino acid, and the subsequent amino acids coupled as in situ generated HOBt or HOAt esters made from appropriate n-α-protected amino acids and HOBt or HOAt by means of DIC in DMF. The acylations were checked by the ninhydrin test performed at 80 oc in order to prevent Fmoc deprotection during the test (Larsen & Holm, “Incomplete Fmoc Deprotection in Solid-phase Synthesis of Peptides,” Int. J. Pept. Protein Res. 43:1-9 (1994), which is hereby incorporated by reference in its entirety).

### Deprotection of the N-α-Amino Protecting Group (Fmoc)

Deprotection of the Fmoc group was performed by treatment with 20% piperidine in DMF (1×5 and 1×10 min.), followed by wash with DMF (5×15 ml, 5 min. each) until no yellow color could be detected after addition of Dhbt-OH to the drained DMF.

### Coupling of Hobt-Esters

3 equivalent (“eq.”) N-α-amino protected amino acid was dissolved in DMF together with 3 eq. HOBt and 3 eq. DIC, and then added to the resin.

### Preformed Symmetrical Anhydride

Six eq. N-α-amino protected amino acid was dissolved in DCM and cooled to 0° C. DIC (3 eq.) was added and the reaction continued for 10 minutes. The solvent was removed in vacuo and the remnant dissolved in DMF. The solution was immediately added to the resin followed by addition of 0.1 eq. of DMAP.

Cleavage of Peptide from Resin with Acid

Peptides were cleaved from the resins by treatment with 95% trifluoroacetic acid (TFA, Riedel-de Häen, Frankfurt, Germany)-water v/v or with 95% TFA and 5% ethandithiol v/v at room temperature for 2 hours. TIS (triisopropylsilan) as well as other scavengers were used instead of ethandithiol or in combination with TIS when necessary. The filtered resins were washed with 95% TFA-water and filtrates and washings evaporated under reduced pressure. The residue was washed with ether and freeze-dried from acetic acid-water. The crude freeze-dried product was analyzed by high-performance liquid chromatography (“HPLC”) and identified by electrospray ionisation mass spectrometry (“ESMS”).

### Batchwise Peptide Synthesis on TentaGel Resin (“PEG-PS”)

TentaGel resin (1 g, 0.22-0.31 mmol/g) was placed in a polyethylene vessel equipped with a polypropylene filter for filtration. The resin was swelled in DMF (15 ml), and treated with 20% piperidine in DMF to secure the presence of non-protonated amino groups on the resin. The resin was drained and washed with DMF until no yellow color could be detected after addition of Dhbt-OH to the drained DMF. HMPA (3 eq.) was coupled as a preformed HOBt-ester as described above and the coupling was continued for 24 hours. The resin was drained and washed with DMF (5×5 ml, 5 min. each) and the acylation checked by the ninhydrin test. The first amino acid was coupled as a preformed symmetrical anhydride as described above. The following amino acids according to the sequence were coupled as preformed Fmoc-protected HOBt esters (3 eq.) as described above. The couplings were continued for 2 hours, unless otherwise specified. The resin was drained and washed with DMF (5×15 ml, 5 min. each) in order to remove excess reagent. All acylations were checked by the ninhydrin test performed at 80° C. After completed synthesis, the peptide-resin was washed with DMF (3×15 ml, 5 min. each), DCM (3×15 ml, 1 min. each), and finally diethyl ether (3×15 ml, 1 min. each), and dried in vacuo.

### Preparative HPLC Conditions

Preparative chromatography was carried out using a VISION Workstation (PerSeptive Biosystem) equipped with an AFC2000 automatic fraction collector/autosampler. VISION-3 software was used for instrument control and data acquisition.

### Column

Chromatography was carried out using the following columns. Kromasil (EKA Chemicals) KR100-10-C8 100 Å, C-8, 10 μm; CER 2230, 250×50, 8 mm, or a VYDAC 218TP101550, 300 Å, C-18, 10-15 μm, 250×50 mm. The buffer system used included A: 0.1% TFA in MQV; and B: 0.085% TFA, 10% MQV, 90% MeCN. Flow rates were 35-40 ml/min and the column temperature was 25° C. UV detection was performed at 215 nm and 280 nm. Suitable gradients were optimized for individual peptides.

### Analytical HPLC Conditions

Gradient HPLC analysis was done using a Hewlett Packard HP 1100 HPLC system consisting of a HP 1100 Quaternary Pump, a HP 1100 Autosampler, a HP 1100 Column Thermostat, and a HP 1100 Multiple Wavelength Detector. Hewlett Packard Chemstation for LC software (rev. A.06.01) was used for instrument control and data acquisition.

For analytical HPLC, different columns were used as appropriate, such as VYDAC 238TP5415, C-18, 5 μm, 300 Å; or a Jupiter, Phenomenex 00E-4053-E0; 5 μm C-18, 300 Å 150×4.6 mm, and others. The buffer system included A: 0.1% TFA in MQV; and B: 0.085% TFA, 10% MQV, 90% MeCN. Flow rates were 1 ml/min. The preferred column temperature was 40° C. UV detection was performed at 215 nm. As above, suitable gradients were optimized for the individual peptides.

### Mass Spectroscopy

The peptides were dissolved in super gradient methanol (Labscan, Dublin, Ireland), Milli-Q water (Millipore, Bedford, Mass.), and formic acid (Merck, Damstadt, Germany) (50:50:0.1 v/v/v) to give concentrations between 1 and 10 μg/ml. The peptide solutions (20 μl) were analysed in positive polarity mode by ESI-TOF-MS using a LCT mass spectrometer (Micromass, Manchester, UK) (accuracy of +/−0.1 m/z).

### General Synthetic Procedure

In all syntheses, dry TentaGel-S-Ram resin (1 g, 0.22-0.31 mmol/g) was placed in a polyethylene vessel equipped with a polypropylene filter for filtration. The resin was swelled in DMF (15 ml), and treated with 20% piperidine in DMF to secure the presence of non-protonated amino groups on the resin. The resin was drained and washed with DMF until no yellow color could be detected after addition of Dhbt-OH to the drained DMF. The amino acids according to the sequence were coupled as preformed Fmoc-protected HOBt esters (3 eq.) as described above.

All couplings were continued for at least 2 hours. The acylations were checked by the ninhydrin test performed at 80° C. as earlier described. After completed synthesis the peptide-resin was washed with DMF (3×15 ml, 1 min. each), DCM (3×15 ml, 1 min. each), and diethyl ether (3×15 ml, 1 min. each), and dried in vacuo. The peptide was then cleaved from the resin as described above and freeze-dried.

After purification using preparative HPLC as described above, the peptide product was collected and the identity of the peptide was confirmed by ES-MS.

### Example 19

### RXPE Mutants

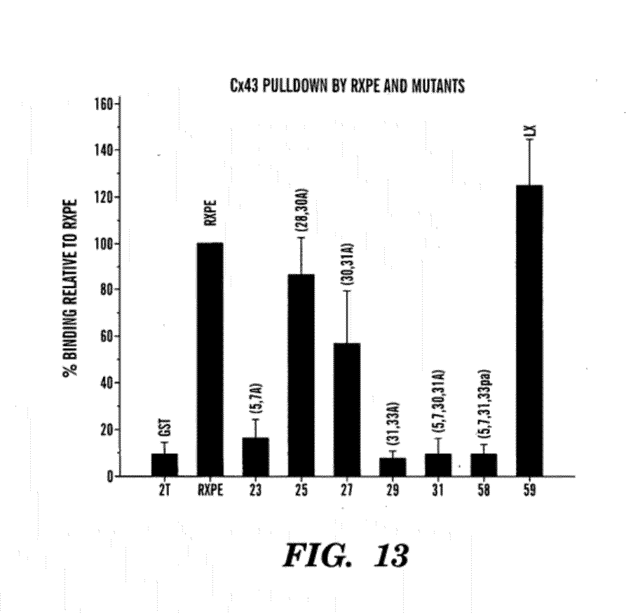

Mutants of RXPE were generated by substituting residues 5, 7, 28, 30, 31, and/or 33 with alanine, as shown in Table 6 (bold indicates substituted residues). These mutants were used in a Cx43 pulldown assay along with RXPE and NL.RXPE. The percent of binding relative to RXPE is shown in FIG. 13.

TABLE 6
RXPE Mutant Peptides

| | Substituted | |
|---|---|---|
| Peptide | Residues | Sequence |
| RXPE | | 5 7 28 30 31 33 |
| | | SDDLRSPQLHNGGGSAVPFYSHSHMVRRKPRNPR |
| | | (SEQ ID NO: 10) |
| #23 | 5, 7 | 5 7 28 30 31 33 |
| | | SDDLASAQLHNGGGSAVPFYSHSHMVRRKPRNPR |
| | | (SEQ ID NO: 34) |
| #25 | 28, 30 | 5 7 28 30 31 33 |
| | | SDDLRSPQLHNGGGSAVPFYSHSHMVRAKARNPR |
| | | (SEQ ID NO: 35) |
| #27 | 30, 31 | 5 7 28 30 31 33 |
| | | SDDLRSPQLHNGGGSAVPFYSHSHMVRRKAANPR |
| | | (SEQ ID NO: 36) |
| #29 | 31, 33 | 5 7 28 30 31 33 |
| | | SDDLRSPQLHNGGGSAVPFYSHSHMVRRKPANAR |
| | | (SEQ ID NO: 37) |
| #31 | 5, 7, 30, 31 | 5 7 28 30 31 33 |
| | | SDDLASAQLHNGGGSAVPFYSHSHMVRRKAANPR |
| | | (SEQ ID NO: 38) |
| #58 | 5, 7, 31, 33 | 5 7 28 30 31 33 |
| | | SDDLASAQLHNGGGSAVPFYSHSHMVRRKPANAR |
| | | (SEQ ID NO: 39) |
| #59 | No linker | 5 7 28 30 31 33 |
| | | SDDLRSPQLHNHMVRRKPRNPR |
| | | (SEQ ID NO: 11) |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A protein or polypeptide comprising the formula A-[Wₘ-A]ₙ wherein
each A is independently a peptide of formula XXXXRXPXXXX wherein each X is independently any amino acid, R is arginine, and P is proline;
each W is independently a linker from about 1 to about 15 amino acids;
each m is independently 0, 1, or 2; and
n is any number from 1 through 5.

2-3. (canceled)

4. The protein or polypeptide according to claim 1, wherein each m is independently 1 or 2.

5-6. (canceled)

7. The protein or polypeptide according to claim 4, wherein the protein or polypeptide comprises an amino acid sequence selected from the group consisting of SEQ ID NO:6, SEQ ID NO:7, SEQ ID NO:8, SEQ ID NO:9, and SEQ ID NO:10.

8. The protein or polypeptide according to claim 7, wherein the protein or polypeptide has an amino acid sequence of SEQ ID NO:10.

9-61. (canceled)


SEQUENCE LISTING (40 sequences)

SEQ ID NO: 1 - protein, 382 aa, human
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 2 - protein, 4 aa, artificial
  Linker Peptide
GGGS

SEQ ID NO: 3 - protein, 11 aa, artificial
  Linker Peptide
AHARVPFYSHS

SEQ ID NO: 4 - protein, 13 aa, artificial
  Linker Peptide
AETHARVPFYSHS

SEQ ID NO: 5 - protein, 8 aa, artificial
  Linker Peptide
AVPFYSHS

SEQ ID NO: 6 - protein, 37 aa, artificial
  RXP-containing peptide
DVPGRDPGYIKGGGSAHARVPFYSHSLNRNRKPSLYQ

SEQ ID NO: 7 - protein, 37 aa, artificial
  RXP-containing peptide
EIQPRSPLMFSGGGSAHARVPFYSHSAKEARWPRAHR

SEQ ID NO: 8 - protein, 37 aa, artificial
  RXP-containing peptide
GIAAREPNSHDGGGSAHARVPFYSHSRDLWRKPAKSL

SEQ ID NO: 9 - protein, 39 aa, artificial
  RXP-containing peptide
WEEPRRPFTMSGGGSAETHARVPFYSHSPMRHRLPGVHL

SEQ ID NO: 10 - protein, 34 aa, artificial
  RXP-containing peptide
SDDLRSPQLHNGGGSAVPFYSHSHMVRRKPRNPR

SEQ ID NO: 11 - protein, 22 aa, artificial
  RXP-containing peptide
SDDLRSPQLHNHMVRRKPRNPR

SEQ ID NO: 12 - protein, 12 aa, artificial
  RXP-containing peptide
GHLHLRVPTLKM

SEQ ID NO: 13 - protein, 12 aa, artificial
  RXP-containing peptide
EFIRSPHSVDWL

SEQ ID NO: 14 - protein, 12 aa, artificial
  RXP-containing peptide
SQSRNPPMPPPR

SEQ ID NO: 15 - protein, 12 aa, artificial
  RXP-containing peptide
RRPPYRVPPKLF

SEQ ID NO: 16 - protein, 12 aa, artificial
  RXP-containing peptide
SLYERHPASTYP

SEQ ID NO: 17 - protein, 12 aa, artificial
  RXP-containing peptide
HTVSRRPLPSSG

SEQ ID NO: 18 - protein, 12 aa, artificial
  RXP-containing peptide
RHTHGNLLRFPP

SEQ ID NO: 19 - protein, 12 aa, artificial
  RXP-containing peptide
RNNLNQTYPERR

SEQ ID NO: 20 - protein, 12 aa, artificial
  RXP-containing peptide
YSLLPVRPVALT

SEQ ID NO: 21 - protein, 12 aa, artificial
  RXP-containing peptide
RKPTQSLPTRLV

SEQ ID NO: 22 - protein, 12 aa, artificial
  RXP-containing peptide
TRRPHKMRSDPL

SEQ ID NO: 23 - protein, 12 aa, artificial
  RXP-containing peptide
TLTWHTKTPVRP

SEQ ID NO: 24 - protein, 12 aa, artificial
  RXP-containing peptide
SRQFLHSLDRLP

SEQ ID NO: 25 - protein, 12 aa, artificial
  RXP-containing peptide
HLHHHLDHRPHR

SEQ ID NO: 26 - protein, 12 aa, artificial
  RXP-containing peptide
QTPYQARLPAVA

SEQ ID NO: 27 - protein, 12 aa, artificial
  RXP-containing peptide
WHPHRHHHLQWD

SEQ ID NO: 28 - protein, 8 aa, artificial
  RXP-containing peptide
RRKPRRKP

SEQ ID NO: 29 - protein, 6 aa, artificial
  RXP-containing peptide
RNPRNP

SEQ ID NO: 30 - protein, 4 aa, artificial
  RXP-containing peptide
RRKP

SEQ ID NO: 31 - protein, 4 aa, artificial
  RXP-containing peptide
RRNP

SEQ ID NO: 32 - protein, 12 aa, artificial
  Peptide sequence from phage library
PRPTMGNLPDVL

SEQ ID NO: 33 - protein, 10 aa, Zonula ocludens
RATLLNVPDL

SEQ ID NO: 34 - protein, 34 aa, artificial
  RXP-containing peptide
SDDLASAQLHNGGGSAVPFYSHSHMVRRKPRNPR

SEQ ID NO: 35 - protein, 34 aa, artificial
  RXP-containing peptide
SDDLRSPQLHNGGGSAVPFYSHSHMVRAKARNPR

SEQ ID NO: 36 - protein, 34 aa, artificial
  RXP-containing peptide
SDDLRSPQLHNGGGSAVPFYSHSHMVRRKAANPR

SEQ ID NO: 37 - protein, 34 aa, artificial
  RXP-containing peptide
SDDLRSPQLHNGGGSAVPFYSHSHMVRRKPANAR

SEQ ID NO: 38 - protein, 34 aa, artificial
  RXP-containing peptide
SDDLASAQLHNGGGSAVPFYSHSHMVRRKAANPR

SEQ ID NO: 39 - protein, 34 aa, artificial
  RXP-containing peptide
SDDLASAQLHNGGGSAVPFYSHSHMVRRKPANAR

SEQ ID NO: 40 - protein, 4 aa, Artificial Sequence
  Recombinant rCx43
GPLG